(19) United States
(12) Patent Application Publication
(10) Pub. No.: US 2025/0249272 A1
(43) Pub. Date: Aug. 7, 2025

(54) SYSTEMS AND METHODS RELATED TO THE TREATMENT OF DYSTONIA

(71) Applicant: Duke University, Durham, NC (US)

(72) Inventors: Noreen Bukhari-Parlakturk, Durham, NC (US); Nicole Calakos, Durham, NC (US); Angel Peterchev, Durham, NC (US)

(21) Appl. No.: 19/047,016

(22) Filed: Feb. 6, 2025

Related U.S. Application Data
(60) Provisional application No. 63/550,238, filed on Feb. 6, 2024.

(51) Int. Cl. A61N 2/00

(52) U.S. Cl. CPC A61N 2/006

(57) ABSTRACT

The present disclosure provides systems and methods related to the treatment of dystonia. In particular, the present disclosure provides systems and methods for identifying cortical regions of a subject's brain associated with dystonia and delivering repetitive transcranial magnetic stimulation (rTMS) to that target cortical region to treat at least one symptom of the dystonia in the subject.

### CROSS-REFERENCE TO RELATED APPLICATIONS

This application claims priority to and the benefit of U.S. Provisional Patent Application No. 63/550,238 filed Feb. 6, 2024, which is incorporated herein by reference in its entirety for all purposes.

### FIELD

The present disclosure provides systems and methods related to the treatment of dystonia. In particular, the present disclosure provides systems and methods for identifying cortical regions of a subject's brain associated with dystonia and delivering repetitive transcranial magnetic stimulation (rTMS) to that target cortical region to treat at least one symptom of the dystonia in the subject.

### BACKGROUND

Repetitive transcranial magnetic stimulation (rTMS) is a noninvasive brain stimulation technology that in a meta-analysis of 27 prior studies showed some benefit in reducing dystonia symptoms. Across these studies, a predictor of benefit was the stimulation site which varied by the dystonia subtype. In dystonias outside of the upper limb, such as cervical dystonia and blepharospasm, some behavioral benefits were reported after TMS to cerebellum and anterior cingulate cortex respectively. In upper limb dystonia, behavioral benefit was reported after TMS to motor-premotor cortex (PMC) or primary somatosensory cortex (PSC). Although PSC only showed benefit in one study, it is noteworthy that the reported behavioral benefit was more enduring (two to three weeks) than any of the nine PMC studies. To best resolve whether one target site is superior, a head-to-head comparison of PMC versus PSC target controlling for the other variables among these studies would be necessary.

In addition to the stimulation site, another predictor of TMS benefit was the stimulation parameters. In upper limb dystonia, behavioral benefit was reported after 1 Hz rTMS, 0.2 Hz rTMS and continuous theta burst (TBS) TMS while in cervical dystonia and blepharospasm, only TBS-TMS and 0.2 Hz rTMS showed benefits respectively. Overall, some behavioral benefit was reported after TMS in adult focal dystonias but varied by dystonia subtypes with key factors being stimulation site and stimulation parameters.

To improve the clinical efficacy of TMS in dystonia, more clinical studies are needed to directly compare the previously effective stimulation sites and parameters within subject. In addition, advances in the understanding of the brain mechanism mediating TMS benefit irrespective of brain disorder is critical to enable future rational optimization of this promising non-invasive therapeutic modality. Collectively, comparative and mechanistic TMS studies are critically needed to guide further refinements in TMS protocols to achieve clinically meaningful and enduring benefits across multiple dystonia subtypes.

### SUMMARY

Embodiments of the present disclosure include methods for treating dystonia. In accordance with these embodiments, the method includes administering repetitive transcranial magnetic stimulation (rTMS) to a subject, wherein the rTMS targets a cortical region of the subject's brain, and wherein administering the rTMS treats at least one symptom of dystonia in the subject.

In some embodiments, the rTMS is administered to the premotor cortex (PMC). In some embodiments, the rTMS is administered to the primary somatosensory cortex (PSC). In some embodiments, the rTMS is administered to the parietal cortex (PC).

In some embodiments, the rTMS is administered at a frequency ranging from about 5 Hz to about 20 Hz.

In some embodiments, the rTMS comprises administering about 10 stimulation trains to about 50 stimulation trains. In some embodiments, the trains are administered at an inter-train interval of about 5 seconds to about 30 seconds.

In some embodiments, the rTMS is administered using a plurality of stimulation blocks, wherein each stimulation block is from about 1 minute to about 10 minutes.

In some embodiments, about 500 pulses to about 2000 pulses are delivered in each stimulation block. In some embodiments each pulse is the same phase (monophasic or biphasic).

In some embodiments, each stimulation block is interleaved with a motor activity block during which the subject performs or imagines performing a motor task.

In some embodiments, the plurality of stimulation blocks range from about 2 stimulation blocks to about 10 stimulation blocks.

In some embodiments, a total of about 1000 pulses to about 50,000 pulses are delivered to the subject during a single treatment.

In some embodiments, the at least one symptom of dystonia that is treated in the subject comprises at least one of muscle tightness, muscle contraction, muscle pain, decreased coordination, abnormal posture, hand spasms, task-related tremor, and/or motor dysfluency.

In some embodiments, administration of the rTMS to the subject improves at least one functional connection in a motor network subregion, thereby treating the at least one symptom of dystonia in the subject.

In some embodiments, the method further comprises assessing neural activity in the subject before and/or after administering the rTMS. In some embodiments, the neural activity is assessed using magnetic resonance imaging (MRI) or functional magnetic resonance imaging (fMRI).

In some embodiments, the method further comprises performing rTMS target selection prior to administering rTMS to the subject, wherein the target selection comprises assessing neural activity of the subject during performance of a motor task. In some embodiments, the neural activity is assessed using magnetic resonance imaging (MRI) or functional magnetic resonance imaging (fMRI).

In some embodiments, the rTMS is administered during more than one treatment session separated by a period of up to about six months. In some embodiments, the rTMS is administered on consecutive days. In some embodiments, rTMS is administered on non-consecutive days. In some embodiments, rTMS is administered on consecutive days ranging from about 2 consecutive days to about consecutive 10 days.

Embodiments of the present disclosure also include a method of treating dystonia in a subject that includes performing repetitive transcranial magnetic stimulation (rTMS) target selection comprising assessing neural activity of the subject during performance of a motor task using functional magnetic resonance imaging; and administering rTMS at a frequency ranging from about 5 to about 20 Hz to a target cortical region of the subject's brain identified using the rTMS target selection, wherein administering the rTMS treats at least one symptom of dystonia in the subject.

### BRIEF DESCRIPTION OF THE DRAWINGS

FIG. 1: TMS target selection and TMS delivery. To select a scalp target for TMS delivery, each subject completed a task-based fMRI at baseline. An individualized scalp target to motor/premotor cortex (PMC) and primary somatosensory cortex (PSC, represented by a red sphere) was then prepared using fMRI and electric field modeling. Subjects received three TMS conditions: 10 Hz rTMS to PSC, 10 Hz rTMS to PMC and Sham rTMS to PMC (total 4,000 pulses). Each TMS condition was delivered over four stimulation blocks during a single visit. To prime the writing motor network during TMS delivery and circumvent concerns that delivering TMS concurrently during a writing task would compromise stimulation accuracy, an interleaved approach of writing task and stimulation blocks was designed. TMS effect was measured using writing behavior and task-based fMRI.

FIG. 2: Individualized targets for rTMS to PMC and PSC in WC. The cortical target for rTMS delivery to left PMC (blue) and left PSC (red) was developed using fMRI and electric field modeling. The final target for PMC-TMS and PSC-TMS for each WC participant is shown overlayed on a standard MNI brain.

FIG. 3: Consort diagram showing the recruitment, inclusion and exclusion number of participants. Total of 34 WC dystonia participants were screened. 24 participants completed fMRI, 14 consented to the TMS study. Of these, 12 participants were randomized and completed the TMS study.

FIG. 4: Schematic overview of study data and analytical pipeline. The diagram illustrates the key datasets collected and analyzed in the study. For each dataset, the statistical models, multiple comparison correction methods, and the corresponding figure and/or table resulting from the dataset are reported. Mixed-effects model for repeated measures (MEMRM) was employed with covariates and covariance structures tailored to each dataset. Correlations were performed using Pearson's R to understand the relationship between peak accelerations, BOLD activity, and functional connectivity. Multiple comparison corrections were performed where applicable.

FIGS. 5A-5E: 10 Hz rTMS to PSC, but not PMC, reduced writing dysfluency in WC. 10 Hz rTMS to PSC significantly reduced a measure of writing dysfluency called peak accelerations compared to sham-TMS in a within-subject analysis in WC participants (FIG. 5A). PMC-TMS did not show significant differences in writing dysfluency compared to sham-TMS. Each data point represents the mean change in peak accelerations for each TMS condition with higher measures representing worsening writing dysfluency. The effect of TMS on right hand dystonia in WC participants was also compared using the clinician-rated scales of Burke Fahn Marsden (BFM) right hand dystonia (FIG. 5B) and Writer's Cramp Rating Scale (WCRS) movement scores (FIG. 5C). TMS effect on WC participants' right-hand disability was reported using the BFM handwriting disability score (FIG. 5D) and Arms Dystonia Disability Scale (ADDS) (FIG. 5E). All rating scales were performed before and after each TMS condition. Each data point on the graph represents a subject's score change (Post-TMS minus Pre-TMS) in the scale. **p<0.01, *p<0.05 after MEMRM test and Tukey-Holm Sidak correction.

FIG. 6A-6B: 10 Hz rTMS to either PMC or PSC decreased subcortical activity in the motor network. Graphs represent mean BOLD activity during the writing and rest blocks after each TMS condition [sham-TMS (green), PMC-TMS (blue) and PSC-TMS (red)](FIG. 6A). The mean BOLD activity is presented for brain regions of the primary somatosensory cortex (PSC, top graph) and caudate (bottom graph, PSC vs. Sham: −0.19, p<0.0001, PMC vs. Sham −0.14, p<0.001, PSC vs. PMC: 0.05, p=0.14, MEMRM and FDR-corrected at p<0.05). Heatmap represents mean BOLD activity during the writing block for subregions of the motor network across the three TMS conditions (FIG. 6B). Asterisks indicate significant difference in mean BOLD activity for PMC-TMS or PSC-TMS compared to sham-TMS (MEMRM and FDR-corrected at p<0.05). SPC: superior parietal cortex, SMA: supplementary motor area, CAU: caudate, PUT: putamen, PAL: pallidum, THL: thalamus, STN: subthalamic nucleus, SN: substantia nigra, CBL-VI: cerebellum, lobule VI and CBL-VIII: cerebellum, lobule VIII.

FIG. 7: 10 Hz rTMS to PSC but not PMC induced changes in cortical-subcortical connectivity in the motor network compared to sham-TMS. The brain model represents TMS induced changes in functional connectivity (FC) in the motor network. Black circles represent brain regions in the motor network and connecting lines indicate FC changes between these regions. Line thickness denotes the direction of FC change (thin line: weakened FC, thick line: strengthened FC). Only FC that were significantly different between PSC-TMS and sham-TMS (p<0.05, MEMRM with FDR correction) are shown. No significant FC changes were observed after PMC-TMS compared to sham-TMS. Detailed FC values for PSC-TMS versus sham-TMS are provided in Table 3. PMC: premotor cortex; PSC: primary somatosensory cortex; SPC: superior parietal cortex; CAU: caudate; PUT: putamen; PAL: pallidum; SN: substantia nigra; R-CBL VI: right cerebellum lobule VI; R-CBL-VIII: right cerebellum lobule VIII.

FIGS. 8A-8B: TMS-induced change in BOLD activity at PSC and SPC correlated with reduced writing dysfluency. Graphs represent the correlation between BOLD activity at primary somatosensory cortex (x-axis) and superior parietal cortex (x-axis) (FIG. 8A) with behavior of peak accelerations (y-axis) for the three TMS conditions (FIG. 8B). Each data point represents the correlation between a WC participant's regional BOLD activity and peak accelerations behavior for each TMS condition. Shaded blue regions represent the confidence region for the fitted lines. A Pearson's correlation (R) is reported for each BOLD activity-behavior correlation and TMS condition.

FIGS. 9A-9B: Reorganization of the motor network connectivity after PSC-TMS correlated with reduced writing dysfluency. Graph represents the correlation between functional connectivity (FC) (x-axis) and peak accelerations behavior (y-axis) for the 12 WC subjects and three TMS conditions (FIG. 9A). Each data point represents a WC participant's FC-behavior relationship. The representative scatter plots are organized by TMS condition and shown for the relationship between PSC-SPC FC to peak accelerations behavior. WC subjects showed no FC-behavior correlation for conditions of sham-TMS (R=0.21) or PMC-TMS (R=0.07). But there is an inverse correlation between PSC-SPC FC and peak accelerations behavior after PSC-TMS (R=−0.69). A heatmap of the mean correlation between FCs in the writing motor network and peak accelerations behavior (FIG. 9B). Each box represents the mean correlation (R) between peak accelerations behavior and a FC for each TMS condition (red box=positive FC-behavior correlation, blue box=negative FC-behavior correlation). Heatmap is reported only for FCs that show R>10.61 for at least one TMS condition (indicated by an asterisk) and compared with the other two TMS conditions. A subset FC-behavior analysis was performed to compare the four FC-behavior relationships that differentiate PSC-TMS from both sham-TMS and PMC-TMS (indicated by “+”). Across the four FC-behavior relationships, strengthening of PSC-SPC connectivity correlated with significant reduction in peak accelerations behavior after PSC-TMS compared to sham-TMS (PSC vs. Sham: −14.6, p=0.075, generalized linear regression, and FDR correction).

FIG. 10A-10E: Minimal deviation between intended and actual TMS scalp targets. Each graph shows the deviation in TMS coil setup defined as the difference between the intended and actual TMS coil parameters for the three TMS conditions. The deviation in TMS coil position is measured in the normal plane (FIG. 10A) and tangential plane (FIG. 10B). The direct distance between the intended TMS coil position and actual TMS coil position are also measured (FIG. 10C). The deviation in TMS coil orientation is also presented in the normal plane (FIG. 10D) and tangential plane (FIG. 10E). Each data point on a graph represents the mean TMS coil deviation across all four TMS blocks for each subject. Data are presented for Sham-rTMS (n=11), 10 Hz rTMS to PMC (n=12), and 10 Hz rTMS to PSC (n=12). No significant differences in TMS coil deviation across the three TMS conditions were observed (MEMRM, p-value <0.05 considered significant).

### DETAILED DESCRIPTION

The present disclosure provides systems and methods relating to the treatment of dystonia. In particular, the present disclosure provides systems and methods for identifying cortical regions of a subject's brain associated with dystonia and delivering repetitive transcranial magnetic stimulation (rTMS) to that target cortical region to treat at least one symptom of the dystonia in the subject.

As described further herein, identifying the optimal stimulation site for engaging and improving the abnormal motor circuit mechanisms in dystonia is a major goal necessary for effectively applying TMS-related interventions for dystonia. Embodiments of the present disclosure used a within-participant, sham-controlled study design in writer's cramp dystonia, coupled with functional neuroimaging and behavior to address these unknowns. Demonstrating that TMS to PSC provides a significant behavioral benefit is a critical first step in moving TMS toward clinical therapy for dystonia. Furthermore, delineating the TMS induced corrective changes in the motor network associated with behavioral improvement in dystonia generates mechanistic hypotheses to guide future therapeutic interventions. The pattern of brain-behavior changes observed after PSC-TMS in this study may also serve as a brain signature for a clinical response to use as a screening tool with other interventional modalities.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “subject” and “patient” generally refer to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject is a human. The subject or patient may be undergoing various forms of treatment (e.g., neuromodulation therapy).

As used herein, “treat,” “treating,” and “treatment” generally refer to reversing, alleviating, or inhibiting the progress of a disease and/or disorder, or one or more symptoms of such disease or disorder, to which such terms apply. Depending on the condition of the subject, the term also refers to preventing a disease or disorder, and includes preventing the onset of a disease or disorder, or preventing the symptoms associated with a disease or disorder. A treatment may be either performed in an acute or chronic way. Treatment and related terms can also refer to reducing the severity of a disease or disorder, or symptoms associated with such disease or disorder, prior to manifestation of the disease or disorder. In some aspects, treating one or more symptoms of a disease or disorder includes providing a degree of relief of one or more symptoms of the disease or disorder.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. METHODS OF TREATMENT

Embodiments of the present disclosure provide systems and methods relating to the treatment of dystonia. In particular, the present disclosure provides systems and methods for identifying cortical regions of a subject's brain associated with dystonia and delivering repetitive transcranial magnetic stimulation (rTMS) to that target cortical region to treat at least one symptom of the dystonia in the subject.

As described further herein, embodiments of the present disclosure compared the efficacy of two TMS cortical sites in reducing dystonic behavior, each previously shown to be beneficial in separate dystonia studies and aimed to identify a TMS-induced brain mechanism underlying the observed behavioral improvement. This led to the development of three findings. First, 10 Hz rTMS to primary somatosensory cortex significantly reduced writing dysfluency compared to Sham and to 10 Hz rTMS at the premotor cortex. These results suggest the clinical potential of 10 Hz rTMS to the PSC for this rare brain disorder. Second, TMS delivered to the same region improved behavior by changing subcortical connectivity in the motor network. Third, results of the present disclosure demonstrated that the intra-cortical connectivity between primary somatosensory and superior parietal cortices are a key predictor for effective stimulation at PSC. Collectively, the findings described herein will guide future refinements in TMS protocols to achieve clinically meaningful and enduring benefits in dystonia.

As described further herein, experimental results indicated that 10 Hz rTMS to PSC significantly reduced writing dysfluency in WC dystonia. The twenty-minute PSC-TMS session showed an effect size of 0.96 compared to Sham. This effect size is among the highest reported for TMS studies in dystonia. The kinematic writing metric was selected based on its high diagnostic performance in a prior exploratory study comparing 22 kinematic writing measures from writer's cramp and healthy volunteers. In that study, across the 22 kinematic measures, peak accelerations showed high sensitivity, specificity, intra-subject reliability, and realistic sample size to power a clinical trial. Importantly, WC participants' baseline measure of peak accelerations also significantly correlated with the clinical scores of BFM right arm dystonia and disability. Results of the present disclosure indicated that three of the four clinical scores showed trends of greater improvement after PSC vs. Sham than PMC vs. Sham (Table 2, Difference column).

Since the cortical gyri for PSC and PMC lie adjacent to the central sulcus, the differential stimulation response to these regions also demonstrated the cortical selectivity of our TMS effect. It is interesting that prior studies reported a behavioral benefit after PMC-TMS. The majority of these studies, however, delivered 1 Hz rTMS to PMC. Therefore, the finding that PSC is a more effective stimulation site than PMC may vary by stimulation frequency. Specifically, this study showed that PSC may be more effective than PMC using 10 Hz rTMS frequency. Additionally, the brain state during TMS delivery may also affect the stimulation site efficacy. As described further herein, TMS was interleaved with writing task to prime the motor network during brain stimulation while in prior studies, TMS was interleaved with periods of rest. Since the brain state during stimulation delivery can change the plasticity inducing mechanism (long term potentiation vs. long term depression), the stimulation at rest in prior studies may have induced a plasticity mechanism that may be different than the present study. Overall, results of the present disclosure expand the range of effective TMS parameters for adult focal hand dystonia and raises the possibility that efficacy of stimulation site may be a function of the stimulation frequency and brain state during TMS delivery.

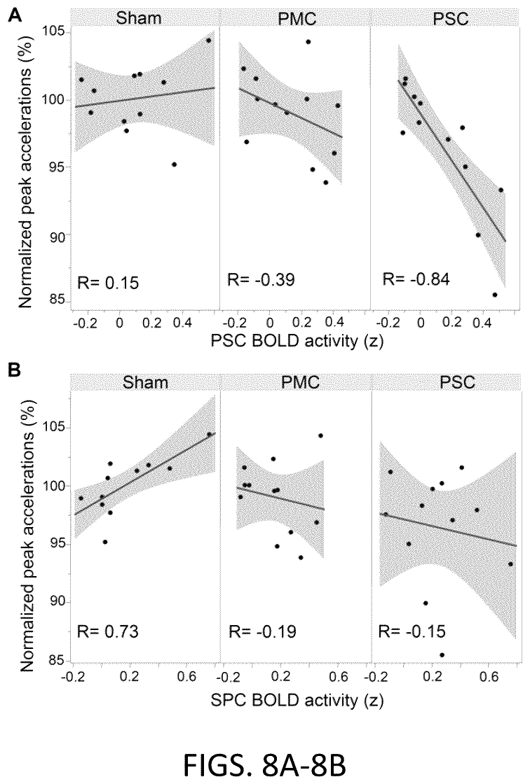

Additionally, results of the present disclosure indicated that 10 Hz rTMS to PSC induced significant changes in subcortical connectivity in the motor network. This was an important study question to guide future refinements in therapeutic applications of TMS in dystonia, where subcortical regions such as basal ganglia and cerebellum play key roles. It is unknown whether active TMS improves behavior by weakening or strengthening brain connections. The results described herein showed that both weakening and strengthening of connections are present resulting in TMS-induced re-organization of the motor network. The strong association between stimulation site (PSC BOLD activity) and writing dysfluency behavior (FIG. 8) further support the potential mechanism that TMS to PSC reduces writing dysfluency behavior. Overall, the present study adds important insights on the TMS induced brain mechanism that contribute to motor behavior benefit in dystonia.

The results of the present disclosure also indicated that the behavioral benefit after PSC-TMS was associated with strengthening of intra-cortical connectivity between somatosensory cortex and superior parietal cortex. The superior parietal cortex is critically important for somatosensory discrimination by providing a mental model of the extremity function. Strengthening of the somatosensory to parietal connectivity may, therefore, be a key mechanism for developing a more accurate mental model of the hand-arm function, which in turn may improve fine motor control and reduce dysfluent writing behavior. Impairment of connectivity between the parietal cortex and somatosensory cortex were previously described in resting-state fMRIs of WC dystonia, and in multimodal imaging analyses of isolated task-specific focal dystonias (WC, musician's dystonia, and spasmodic dysphonia).

Taken together, the results of the present disclosure represent the first interventional study to identify relationships between brain connectivity and dystonic behavior in the sham-TMS condition to inform the pathophysiology. Results of the present disclosure demonstrated that three of the four brain-behavior connections that differentiate the effective stimulation site of PSC from noneffective stimulation sites of PMC and sham conditions are connections to or within the basal ganglia regions (caudate-substantia nigra, putamen-pallidum, putamen-substantia nigra). Furthermore, the present disclosure demonstrates that the brain connectivity pattern of subregions of the motor network are responsive to change in a direction that improves behavior after PSC-TMS compared to sham-TMS. This brain connectivity to behavior patterns can be developed into a screening tool for future interventional trials in dystonia.

The brain-behavior relationships described herein provide greater insight into the cerebellum's role in dystonia, identifying cortico-cerebellar circuitry as clinically relevant. WC participants after sham-TMS showed direct correlation between cortico-cerebellar connectivity and writing dysfluency, a relationship that was absent after PSC-TMS. Results of the present disclosure further support a causal role of this circuitry because TMS to PSC leads to less writing dysfluency and a significant loss of correlation between cortico-cerebellar connectivity and writing dysfluency. These results identify key associations between subcortical-cerebellum and intra-cerebellar connectivity and behavior of writing dysfluency.

Thus, embodiments of the present disclosure used a within-participant, sham-controlled study design in writer's cramp dystonia, coupled with functional neuroimaging and behavior to address these unknowns. Demonstrating that TMS to PSC provides a significant behavioral benefit is a critical first step in moving TMS toward clinical therapy for dystonia. Furthermore, delineating the TMS induced corrective changes in the motor network associated with behavioral improvement in dystonia generates mechanistic hypotheses to guide future therapeutic interventions. The pattern of brain-behavior changes observed after PSC-TMS in this study may also serve as a brain signature for a clinical response to use as a screening tool with other interventional modalities.

In accordance with the above, embodiments of the present disclosure include methods for treating dystonia. In some embodiments, the methods include administering repetitive transcranial magnetic stimulation (rTMS) to a subject in a manner that targets a cortical region of the subject's brain, such that administering the rTMS treats at least one symptom of dystonia in the subject. Additionally, as described further herein, embodiments of the present disclosure include a method for treating dystonia in a subject that includes performing rTMS target selection by assessing neural activity of the subject during performance of a motor task using functional magnetic resonance imaging (fMRI), and administering rTMS according to the parameters described herein, such that administration of the rTMS treats at least one symptom of dystonia in the subject.

In some embodiments, the rTMS is administered to the premotor cortex (PMC), the primary somatosensory cortex (PSC), and/or the parietal cortex (PC). In some embodiments, the rTMS is administered to the premotor cortex (PMC). In some embodiments, the rTMS is administered to the primary somatosensory cortex (PSC). In some embodiments, the rTMS is administered to the parietal cortex (PC).

In some embodiments, the rTMS is administered at a frequency that has been determined to target a cortical region of a subject's brain and to alleviate at least one symptom of dystonia in the subject. Although one of ordinary skill in the art would understand that different treatment parameters and different cortical regions of a subject's brain require different rTMS frequencies, in some embodiments, the rTMS according to the methods of the present disclosure can be administered at a frequency ranging from about 1 Hz to about 20 Hz. In some embodiments, rTMS is administered at a frequency ranging from about 2 Hz to about 20 Hz. In some embodiments, rTMS is administered at a frequency ranging from about 3 Hz to about 20 Hz. In some embodiments, rTMS is administered at a frequency ranging from about 4 Hz to about 20 Hz. In some embodiments, rTMS is administered at a frequency ranging from about 5 Hz to about 20 Hz. In some embodiments, rTMS is administered at a frequency ranging from about 6 Hz to about 20 Hz. In some embodiments, rTMS is administered at a frequency ranging from about 7 Hz to about 20 Hz. In some embodiments, rTMS is administered at a frequency ranging from about 8 Hz to about 20 Hz. In some embodiments, rTMS is administered at a frequency ranging from about 9 Hz to about 20 Hz. In some embodiments, rTMS is administered at a frequency ranging from about 10 Hz to about 20 Hz. In some embodiments, rTMS is administered at a frequency ranging from about 11 Hz to about 20 Hz. In some embodiments, rTMS is administered at a frequency ranging from about 12 Hz to about 20 Hz. In some embodiments, rTMS is administered at a frequency ranging from about 13 Hz to about 20 Hz. In some embodiments, rTMS is administered at a frequency ranging from about 14 Hz to about 20 Hz. In some embodiments, rTMS is administered at a frequency ranging from about 15 Hz to about 20 Hz. In some embodiments, rTMS is administered at a frequency ranging from about 16 Hz to about 20 Hz. In some embodiments, rTMS is administered at a frequency ranging from about 17 Hz to about 20 Hz. In some embodiments, rTMS is administered at a frequency ranging from about 18 Hz to about 20 Hz. In some embodiments, rTMS is administered at a frequency ranging from about 19 Hz to about 20 Hz. In some embodiments, rTMS is administered at a frequency ranging from about 1 Hz to about 19 Hz. In some embodiments, rTMS is administered at a frequency ranging from about 1 Hz to about 18 Hz. In some embodiments, rTMS is administered at a frequency ranging from about 1 Hz to about 17 Hz. In some embodiments, rTMS is administered at a frequency ranging from about 1 Hz to about 16 Hz. In some embodiments, rTMS is administered at a frequency ranging from about 1 Hz to about 15 Hz. In some embodiments, rTMS is administered at a frequency ranging from about 1 Hz to about 14 Hz. In some embodiments, rTMS is administered at a frequency ranging from about 1 Hz to about 13 Hz. In some embodiments, rTMS is administered at a frequency ranging from about 1 Hz to about 12 Hz. In some embodiments, rTMS is administered at a frequency ranging from about 1 Hz to about 11 Hz. In some embodiments, rTMS is administered at a frequency ranging from about 1 Hz to about 10 Hz. In some embodiments, rTMS is administered at a frequency ranging from about 1 Hz to about 9 Hz. In some embodiments, rTMS is administered at a frequency ranging from about 1 Hz to about 8 Hz. In some embodiments, rTMS is administered at a frequency ranging from about 1 Hz to about 7 Hz. In some embodiments, rTMS is administered at a frequency ranging from about 1 Hz to about 6 Hz. In some embodiments, rTMS is administered at a frequency ranging from about 1 Hz to about 5 Hz. In some embodiments, rTMS is administered at a frequency ranging from about 1 Hz to about 4 Hz. In some embodiments, rTMS is administered at a frequency ranging from about 1 Hz to about 3 Hz. In some embodiments, rTMS is administered at a frequency ranging from about 1 Hz to about 2 Hz. In some embodiments, rTMS is administered at a frequency ranging from about 5 Hz to about 15 Hz. In some embodiments, rTMS is administered at a frequency ranging from about 5 Hz to about 10 Hz. In some embodiments, rTMS is administered at a frequency ranging from about 10 Hz to about 15 Hz. In some embodiments, rTMS is administered at a frequency ranging from about 4 Hz to about 16 Hz. In some embodiments, rTMS is administered at a frequency ranging from about 8 Hz to about 12 Hz. In some embodiments, rTMS is administered at a frequency ranging from about 9 Hz to about 11 Hz. In some embodiments, rTMS is administered at a frequency of about 1 Hz, about 2 Hz, about 3 Hz, about 4 Hz, about 5 Hz, about 6 Hz, about 7 Hz, about 8 Hz, about 9 Hz, about 10 Hz, about 11 Hz, about 12 Hz, about 13 Hz, about 14 Hz, about 15 Hz, about 16 Hz, about 17 Hz, about 18 Hz, about 19 Hz, or about 20 Hz.

As provided herein, rTMS can be administered to a subject in need thereof by delivering trains of pulses, or stimulation trains, which are a series of magnetic pulses delivered to the brain of a subject to modulate activity. In some embodiments, the methods of the present disclosure include administering rTMS comprising about 10 stimulation trains to about 50 stimulation trains. In some embodiments, the methods of the present disclosure include administering rTMS comprising about 10 stimulation trains to about 45 stimulation trains. In some embodiments, the methods of the present disclosure include administering rTMS comprising about 10 stimulation trains to about 40 stimulation trains. In some embodiments, the methods of the present disclosure include administering rTMS comprising about 10 stimulation trains to about 35 stimulation trains. In some embodiments, the methods of the present disclosure include administering rTMS comprising about 10 stimulation trains to about 30 stimulation trains. In some embodiments, the methods of the present disclosure include administering rTMS comprising about 10 stimulation trains to about 25 stimulation trains. In some embodiments, the methods of the present disclosure include administering rTMS comprising about 10 stimulation trains to about 20 stimulation trains. In some embodiments, the methods of the present disclosure include administering rTMS comprising about 10 stimulation trains to about 15 stimulation trains. In some embodiments, the methods of the present disclosure include administering rTMS comprising about 15 stimulation trains to about 50 stimulation trains. In some embodiments, the methods of the present disclosure include administering rTMS comprising about 20 stimulation trains to about 50 stimulation trains. In some embodiments, the methods of the present disclosure include administering rTMS comprising about 25 stimulation trains to about 50 stimulation trains. In some embodiments, the methods of the present disclosure include administering rTMS comprising about 30 stimulation trains to about 50 stimulation trains. In some embodiments, the methods of the present disclosure include administering rTMS comprising about 35 stimulation trains to about 50 stimulation trains. In some embodiments, the methods of the present disclosure include administering rTMS comprising about 40 stimulation trains to about 50 stimulation trains. In some embodiments, the methods of the present disclosure include administering rTMS comprising about 45 stimulation trains to about 50 stimulation trains. In some embodiments, the methods of the present disclosure include administering rTMS comprising about 15 stimulation trains to about 45 stimulation trains. In some embodiments, the methods of the present disclosure include administering rTMS comprising about 15 stimulation trains to about 30 stimulation trains. In some embodiments, the methods of the present disclosure include administering rTMS comprising about 20 stimulation trains to about 40 stimulation trains. In some embodiments, the methods of the present disclosure include administering rTMS comprising about 25 stimulation trains to about 35 stimulation trains. In some embodiments, the methods of the present disclosure include administering rTMS comprising about 10 stimulation trains, about 15 stimulation trains, about 20 stimulation trains, about 25 stimulation trains, about 30 stimulation trains, about 35 stimulation trains, about 40 stimulation trains, or about 45 stimulation trains. One of ordinary skill in the art would understand based on the present disclosure that the particular number of stimulation trains administered to a subject according to the methods of the present disclosure will vary depending on a number of factors, including but not limited to, different rTMS treatment parameters and different cortical regions being targeted.

As described further herein, the methods of the present disclosure include administering rTMS using stimulation trains applied at an inter-train interval of about 5 seconds to about 30 seconds. In some embodiments, stimulation trains are administered at an inter-train interval of about 5 seconds to about 25 seconds. In some embodiments, stimulation trains are administered at an inter-train interval of about 5 seconds to about 20 seconds. In some embodiments, stimulation trains are administered at an inter-train interval of about 5 seconds to about 15 seconds. In some embodiments, stimulation trains are administered at an inter-train interval of about 5 seconds to about 10 seconds. In some embodiments, stimulation trains are administered at an inter-train interval of about 10 seconds to about 30 seconds. In some embodiments, stimulation trains are administered at an inter-train interval of about 15 seconds to about 30 seconds. In some embodiments, stimulation trains are administered at an inter-train interval of about 20 seconds to about 30 seconds. In some embodiments, stimulation trains are administered at an inter-train interval of about 25 seconds to about 30 seconds. In some embodiments, stimulation trains are administered at an inter-train interval of about 10 seconds to about 25 seconds. In some embodiments, stimulation trains are administered at an inter-train interval of about 15 seconds to about 20 seconds. In some embodiments, stimulation trains are administered at an inter-train interval of about 20 seconds to about 25 seconds. In some embodiments, stimulation trains are administered at an inter-train interval of about 5 seconds, about 6 seconds, about 7 seconds, about 8 seconds, about 9 seconds, about 10 seconds, 11 seconds, about 12 seconds, about 13 seconds, about 14 seconds, about 15 seconds, about 16 seconds, 17 seconds, about 18 seconds, about 19 seconds, about 20 seconds, about 21 seconds, about 22 seconds, 23 seconds, about 24 seconds, about 25 seconds, about 26 seconds, about 27 seconds, about 28 seconds, 29 seconds, or about 30 seconds. One of ordinary skill in the art would understand based on the present disclosure that the particular length of the inter-train interval will vary depending on a number of factors, including but not limited to, different rTMS treatment parameters and different cortical regions being targeted.

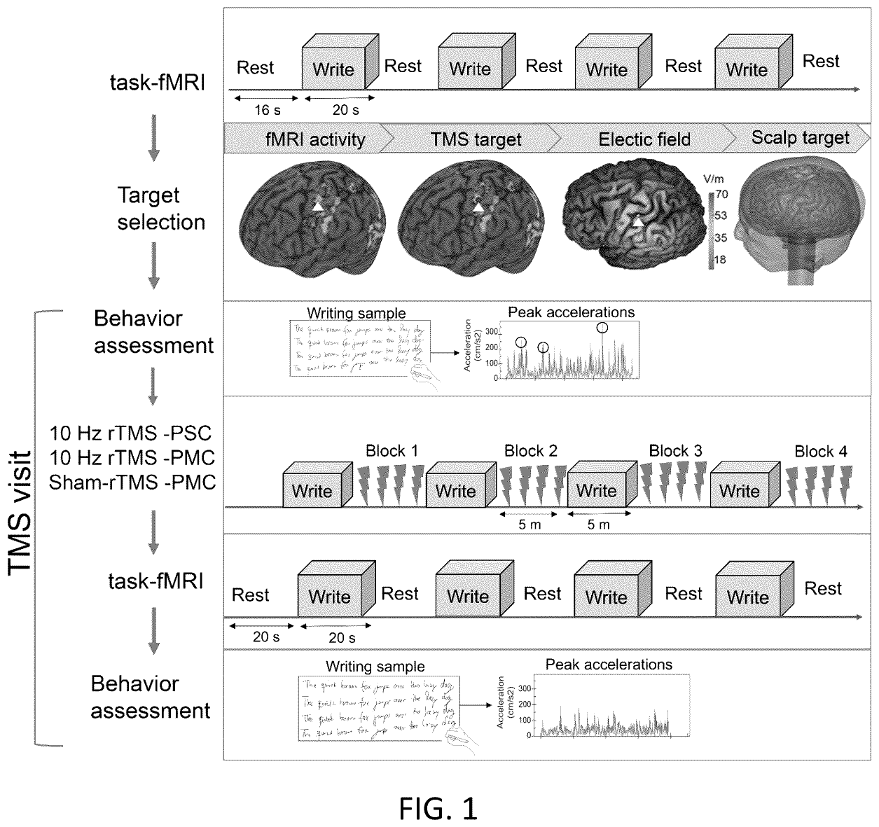

As described further herein, embodiments of the present disclosure include rTMS that is administered using a plurality of stimulation blocks (see, e.g., FIG. 1). In some embodiments, rTMS is administered using a stimulation block(s) interleaved with a motor activity block(s) during which the subject performs or imagines performing a motor task (see, e.g., FIG. 1). In accordance with these embodiments, each stimulation block is from about 1 minute to about 10 minutes. In some embodiments, each stimulation block is from about 1 minute to about 9 minutes. In some embodiments, each stimulation block is from about 1 minute to about 8 minutes. In some embodiments, each stimulation block is from about 1 minute to about 7 minutes. In some embodiments, each stimulation block is from about 1 minute to about 6 minutes. In some embodiments, each stimulation block is from about 1 minute to about 5 minutes. In some embodiments, each stimulation block is from about 1 minute to about 4 minutes. In some embodiments, each stimulation block is from about 1 minute to about 3 minutes. In some embodiments, each stimulation block is from about 1 minute to about 2 minutes. In some embodiments, each stimulation block is from about 2 minutes to about 10 minutes. In some embodiments, each stimulation block is from about 3 minutes to about 10 minutes. In some embodiments, each stimulation block is from about 4 minutes to about 10 minutes. In some embodiments, each stimulation block is from about 5 minutes to about 10 minutes. In some embodiments, each stimulation block is from about 6 minutes to about 10 minutes. In some embodiments, each stimulation block is from about 7 minutes to about 10 minutes. In some embodiments, each stimulation block is from about 8 minutes to about 10 minutes. In some embodiments, each stimulation block is from about 9 minutes to about 10 minutes. In some embodiments, each stimulation block is from about 2 minutes to about 8 minutes. In some embodiments, each stimulation block is from about 3 minutes to about 7 minutes. In some embodiments, each stimulation block is from about 4 minutes to about 8 minutes. In some embodiments, each stimulation block is from about 5 minutes to about 9 minutes. In some embodiments, each stimulation block is from about 3 minutes to about 6 minutes. In some embodiments, each stimulation block is about 1 minute, about 2 minutes, about 3 minutes, about 4 minutes, about 5 minutes, about 6 minutes, about 7 minutes, about 8 minutes, about 9 minutes, or about 10 minutes. One of ordinary skill in the art would understand based on the present disclosure that the particular length of the stimulation block will vary depending on a number of factors, including but not limited to, different rTMS treatment parameters and different cortical regions being targeted.

As described further herein, embodiments of the present disclosure include rTMS that is administered using a plurality of pulses within each stimulation train. In some embodiments each pulse is the same phase (e.g., monophasic or biphasic). In accordance with these embodiments, about 500 pulses to about 2000 pulses are delivered in each stimulation block. In some embodiments, about 500 pulses to about 1750 pulses are delivered in each stimulation block. In some embodiments, about 500 pulses to about 1500 pulses are delivered in each stimulation block. In some embodiments, about 500 pulses to about 1250 pulses are delivered in each stimulation block. In some embodiments, about 500 pulses to about 1000 pulses are delivered in each stimulation block. In some embodiments, about 500 pulses to about 750 pulses are delivered in each stimulation block. In some embodiments, about 750 pulses to about 2000 pulses are delivered in each stimulation block. In some embodiments, about 1000 pulses to about 2000 pulses are delivered in each stimulation block. In some embodiments, about 1250 pulses to about 2000 pulses are delivered in each stimulation block. In some embodiments, about 1500 pulses to about 2000 pulses are delivered in each stimulation block. In some embodiments, about 1750 pulses to about 2000 pulses are delivered in each stimulation block. In some embodiments, about 750 pulses to about 1750 pulses are delivered in each stimulation block. In some embodiments, about 1000 pulses to about 1500 pulses are delivered in each stimulation block. In some embodiments, about 500 pulses to about 2000 pulses are delivered in each stimulation block. In some embodiments, about 500 pulses, about 600 pulses, about 700 pulses, about 800 pulses, about 900 pulses, about 1000 pulses, about 1100 pulses, about 1200 pulses, about 1300 pulses, about 1400 pulses, about 1500 pulses, about 1600 pulses, about 1700 pulses, about 1800 pulses, about 1900 pulses, or about 2000 pulses are delivered in each stimulation block. One of ordinary skill in the art would understand based on the present disclosure that the particular number pulses within each stimulation train will vary depending on a number of factors, including but not limited to, different rTMS treatment parameters and different cortical regions being targeted.

In some embodiments, rTMS is administered using a stimulation block(s) interleaved with a motor activity block(s) during which the subject performs or imagines performing a motor task (see, e.g., FIG. 1). In accordance with these embodiments, a plurality of stimulation blocks ranges from about 2 stimulation blocks to about 10 stimulation blocks. In some embodiments, a plurality of stimulation blocks ranges from about 2 stimulation blocks to about 8 stimulation blocks. In some embodiments, a plurality of stimulation blocks ranges from about 2 stimulation blocks to about 6 stimulation blocks. In some embodiments, a plurality of stimulation blocks ranges from about 2 stimulation blocks to about 4 stimulation blocks. In some embodiments, a plurality of stimulation blocks ranges from about 4 stimulation blocks to about 10 stimulation blocks. In some embodiments, a plurality of stimulation blocks ranges from about 6 stimulation blocks to about 10 stimulation blocks. In some embodiments, a plurality of stimulation blocks ranges from about 8 stimulation blocks to about 10 stimulation blocks. In some embodiments, a plurality of stimulation blocks ranges from about 3 stimulation blocks to about 7 stimulation blocks. In some embodiments, a plurality of stimulation blocks ranges from about 5 stimulation blocks to about 8 stimulation blocks. In some embodiments, a plurality of stimulation blocks includes about 2 stimulation blocks, about 3 stimulation blocks, about 4 stimulation blocks, about 5 stimulation blocks, about 6 stimulation blocks, about 7 stimulation blocks, about 8 stimulation blocks, about 9 stimulation blocks, or about 10 stimulation blocks. One of ordinary skill in the art would understand based on the present disclosure that the particular number stimulation blocks will vary depending on a number of factors, including but not limited to, different rTMS treatment parameters and different cortical regions being targeted.

As described further herein, embodiments of the present disclosure include systems and methods for identifying cortical regions of a subject's brain associated with dystonia and delivering repetitive transcranial magnetic stimulation (rTMS) to that target cortical region to treat at least one symptom of the dystonia in the subject. As used herein, “treat,” “treating,” and “treatment” generally refer to reversing, alleviating, or inhibiting the progress of a disease and/or disorder, or one or more symptoms of such disease or disorder, to which such terms apply. Depending on the condition of the subject, the term also refers to preventing a disease or disorder, and includes preventing the onset of a disease or disorder, or preventing the symptoms associated with a disease or disorder. A treatment may be either performed in an acute or chronic way. Treatment and related terms can also refer to reducing the severity of a disease or disorder, or symptoms associated with such disease or disorder, prior to manifestation of the disease or disorder. In some aspects, treating one or more symptoms of a disease or disorder includes providing a degree of relief of one or more symptoms of the disease or disorder.

As described further herein, at least one symptom of dystonia that can be treated in the subject using the methods of the present disclosure includes muscle tightness, muscle contraction, muscle pain, decreased coordination, abnormal posture, hand spasms, task-related tremor, and/or motor dysfluency. In some embodiments, administration of the rTMS to the subject improves at least one functional connection in a motor network subregion, thereby treating the at least one symptom of dystonia in the subject. In some embodiments, the method further comprises assessing neural activity in the subject before and/or after administering the rTMS. In some embodiments, the neural activity is assessed using magnetic resonance imaging (MRI). In some embodiments, the neural activity is assessed using functional magnetic resonance imaging (fMRI). In some embodiments, the method further comprises performing rTMS target selection prior to administering rTMS to the subject, wherein the target selection comprises assessing neural activity of the subject during performance of a motor task. In some embodiments, the neural activity is assessed using MRI or fMRI.

In accordance with these embodiments, the rTMS treatment can be administered to a subject during more than one treatment session. In some embodiments, the rTMS is administered on consecutive days. In some embodiments, rTMS is administered on non-consecutive days. In some embodiments, rTMS is administered on consecutive days ranging from about 2 consecutive days to about consecutive 20 days. In some embodiments, rTMS is administered on consecutive days ranging from about 2 consecutive days to about consecutive 15 days. In some embodiments, rTMS is administered on consecutive days ranging from about 2 consecutive days to about consecutive 10 days. In some embodiments, rTMS is administered on consecutive days ranging from about 2 consecutive days to about consecutive 10 days. In some embodiments, rTMS is administered on consecutive days ranging from about 2 consecutive days to about consecutive 5 days. In some embodiments, rTMS is administered on consecutive days ranging from about 4 consecutive days to about consecutive 20 days. In some embodiments, rTMS is administered on consecutive days ranging from about 6 consecutive days to about consecutive 20 days. In some embodiments, rTMS is administered on consecutive days ranging from about 8 consecutive days to about consecutive 20 days. In some embodiments, rTMS is administered on consecutive days ranging from about 10 consecutive days to about consecutive 20 days. In some embodiments, rTMS is administered on consecutive days ranging from about 12 consecutive days to about consecutive 20 days. In some embodiments, rTMS is administered on consecutive days ranging from about 14 consecutive days to about consecutive 20 days. In some embodiments, rTMS is administered on consecutive days ranging from about 16 consecutive days to about consecutive 20 days. In some embodiments, rTMS is administered on consecutive days ranging from about 18 consecutive days to about consecutive 20 days. In some embodiments, rTMS is administered on consecutive days ranging from about 4 consecutive days to about consecutive 12 days. In some embodiments, rTMS is administered on consecutive days ranging from about 5 consecutive days to about consecutive 15 days. In some embodiments, rTMS is administered on consecutive days ranging from about 6 consecutive days to about consecutive 10 days. In some embodiments, rTMS is administered on consecutive days ranging from about 8 consecutive days to about consecutive 12 days.

In some embodiments, more than one treatment session can be separated by a period of hours, up to a period of about six months, depending on the needs of the subject and the efficacy of the treatment. In some embodiments, rTMS treatment sessions of the present disclosure can be separated by hours, days, weeks, months, or years. In some embodiments, rTMS treatment sessions of the present disclosure are separated by at least about one day to about one week. In some embodiments, rTMS treatment sessions of the present disclosure are separated by at least about one week to about one month. In some embodiments, rTMS treatment sessions of the present disclosure are separated by at least about one month to about six months.

As described further herein, embodiments of the present disclosure include delivering total of about 1000 pulses to about 50,000 pulses to a subject during a single rTMS treatment session (e.g., on a single day). In some embodiments, a total of about 1,000 pulses to about 45,000 pulses are delivered to the subject during a single treatment. In some embodiments, a total of about 1,000 pulses to about 8,000 pulses are delivered to the subject during a single treatment. In some embodiments, a total of about 1,000 pulses to about 40,000 pulses are delivered to the subject during a single treatment. In some embodiments, a total of about 1,000 pulses to about 6,000 pulses are delivered to the subject during a single treatment. In some embodiments, a total of about 1,000 pulses to about 35,000 pulses are delivered to the subject during a single treatment. In some embodiments, a total of about 1,000 pulses to about 20,000 pulses are delivered to the subject during a single treatment. In some embodiments, a total of about 1,000 pulses to about 15,000 pulses are delivered to the subject during a single treatment. In some embodiments, a total of about 1,000 pulses to about 10,000 pulses are delivered to the subject during a single treatment. In some embodiments, a total of about 5,000 pulses to about 50,000 pulses are delivered to the subject during a single treatment. In some embodiments, a total of about 10,000 pulses to about 50,000 pulses are delivered to the subject during a single treatment. In some embodiments, a total of about 15,000 pulses to about 50,000 pulses are delivered to the subject during a single treatment. In some embodiments, a total of about 20,000 pulses to about 50,000 pulses are delivered to the subject during a single treatment. In some embodiments, a total of about 25,000 pulses to about 50,000 pulses are delivered to the subject during a single treatment. In some embodiments, a total of about 30,000 pulses to about 50,000 pulses are delivered to the subject during a single treatment. In some embodiments, a total of about 35,000 pulses to about 50,000 pulses are delivered to the subject during a single treatment. In some embodiments, a total of about 40,000 pulses to about 50,000 pulses are delivered to the subject during a single treatment. In some embodiments, a total of about 45,000 pulses to about 50,000 pulses are delivered to the subject during a single treatment. In some embodiments, a total of about 10,000 pulses to about 20,000 pulses are delivered to the subject during a single treatment. In some embodiments, a total of about 15,000 pulses to about 45,000 pulses are delivered to the subject during a single treatment. In some embodiments, a total of about 20,000 pulses to about 40,000 pulses are delivered to the subject during a single treatment. In some embodiments, a total of about 10,000 pulses, about 30,000 pulses, about 10,000 pulses, about 12,000 pulses, about 14,000 pulses, about 66,000 pulses, about 18,000 pulses, about 20,000 pulses, about 25,000 pulses, about 30,000 pulses, about 35,000 pulses, or about 40,000 pulses are delivered to the subject during a single treatment. One of ordinary skill in the art would understand based on the present disclosure that the particular number pulses administered during a particular treatment will vary depending on a number of factors, including but not limited to, different rTMS treatment parameters and different cortical regions being targeted.

### 3. NEUROMODULATION SYSTEMS

Embodiments of the present disclosure also include neuromodulation systems for performing the methods described herein. Although any neuromodulation system can be used, preferred systems includes those that are well-suited for performing repetitive transcranial magnetic stimulation (rTMS). As described further herein, rTMS is a non-invasive neurostimulation technique that employs pulsed magnetic fields to modulate the electrical activity of the brain. The rTMS systems of the present disclosure can include a high-current pulse generator that produces a magnetic field, which is transmitted through a stimulation coil strategically positioned near the subject's head. These magnetic pulses generate an electrical field within the brain, capable of depolarizing superficial axons and activating cortical neural networks. Typically, an rTMS device generates a magnetic field ranging from 1.5 to 3 Tesla in brief pulses lasting a fraction of a millisecond, similar to the operation of magnetic resonance imaging (MRI) devices. The extent of neuromodulation achieved through rTMS is contingent upon various parameters, including the type, orientation, and location of the coil, the distance from the brain, the waveform of the magnetic pulse, and the stimulation intensity, frequency, and pattern. One of the notable advantages of rTMS is that it does not require anesthesia, and the majority of adverse effects experienced by patients are transient and mild. A common protocol for treating depression involves 20-40 minute sessions of high-frequency rTMS targeted at the left dorsolateral prefrontal cortex, with a total of 3,000-6,000 pulses administered per session, occurring five days per week over a duration of four to eight weeks. Earplugs are often utilized during treatment to minimize auditory discomfort.

The action of rTMS is predicated on its ability to create an electrical field within the brain, which modulates cortical activity. When stimulation is delivered at an intensity relative to the motor threshold—the threshold necessary to evoke an involuntary muscle contraction—there is a depolarization of superficial axons, leading to current flow across the superficial cortex of the target area. Notably, cortical tracts within the gyrus that are perpendicular to the coil are the most likely to undergo depolarization. Research indicates that the effects of rTMS may be similar to those resulting from electroconvulsive therapy (ECT), such as increased monoamine turnover, elevated levels of brain-derived neurotrophic factor, and modification of brain's synaptic connections. The impact on cortical excitability is characterized by mixed excitatory and inhibitory effects, with low-frequency stimulation generally inducing inhibition and high-frequency stimulation inducing excitation, potentially through the inhibition of inhibitory actions rather than direct enhancement.

In accordance with the above, an rTMS system includes several key components that work together to deliver targeted neuromodulation therapy. The primary components include a pulse generator. For example, a high-current pulse generator produces the electrical energy required to create the magnetic pulses necessary for stimulation. It is responsible for controlling the frequency, intensity, and duration of the pulses delivered. Another component is the magnetic coil. The magnetic coil is the component that generates the magnetic field. It is typically made in various shapes, such as a figure-8 or round coil, allowing for different stimulation depths and focality. The coil is placed near the patient's head and directly influences the targeted brain regions. Another component is the control unit. The control unit serves as the interface for the operator to program the treatment parameters. It includes a user-friendly display and controls to adjust settings such as pulse frequency, intensity, and the number of pulses to be delivered during a session. The control unit may also include safety features and monitoring systems to ensure patient safety. Another component is the subject/patient interface. This component includes the apparatus used to position the magnetic coil accurately over the desired area of the patient's scalp. It may involve adjustable supports or specialized headgear to ensure comfort and stability during treatment sessions. Another component is the safety mechanism. Safety mechanisms are integral to the rTMS system, ensuring that the treatment adheres to established medical guidelines. These mechanisms can include auditory alarms, emergency shut-off functions, and monitoring systems to assess the patient's physiological responses during stimulation. In some embodiments, the rTMS systems of the present disclosure also include various accessories, such as but not limited to, earplugs to mitigate sound discomfort caused by the magnetic pulses, as well as a computer or software for data logging and treatment planning.

### 4. MATERIALS AND METHODS

Study design: The study was a double-blind sham-controlled cross-over design with data collected at Duke University Hospital between September 2018 and September 2022. The study was approved by the Duke Health Institutional Review Board (IRB #0094131), registered on clinicaltrials.gov (NCT 06422104) and performed in accordance with the Declaration of Helsinki. All participants gave written informed consent prior to any study participation. Inclusion criteria were adults (>18 years), diagnosed with isolated right-hand dystonia by a Movement Disorder Specialist, more than three months from the last botulinum toxin injection, more than one month from trihexyphenidyl medication, and able to sign an informed consent form. Exclusion criteria were any contraindications to receiving MRI or TMS.

MRI data acquisition and preprocessing: All study participants completed a brain imaging scan pre-TMS and those who consented to TMS also completed it after each TMS visit. All anatomical and functional imaging data was collected on a 3 Tesla GE scanner equipped with an 8-channel head coil. The anatomical MRI scan was acquired using T1-weighted echo-planar sequence with the following parameters: voxel size: 256×256 matrix, repetition time (TR)=7.316 ms, echo time (TE)=3.036 ms, field of view (FOV)=25.6 mm, 1 mm slice thickness. During fMRI sequences, participants copied holo-alphabetic sentences on an MRI-compatible writing tablet. The sentence writing was performed in a block design alternated by rest blocks (FIG. 1, task-fMRI panels). The CIGAL software presented visual writing instructions and recorded participants' movements during the fMRI scan. Pre-TMS MRI: Functional echo-planar images were acquired using the following parameters: voxel size: 3.5×3.5×4.0 mm³, flip angle 90°, TR=2 s, TE=30 ms, FOV=22 mm for 37 interleaved slices in ascending order, writing block: 20 s, rest block: 16 s, total: 12 blocks per fMRI. Post-TMS fMRI: Functional echo-planar images were acquired with the following parameters: voxel size: 2.0×2.0×3.0 mm³, flip angle 90°, TR=2.826 s, TE=25 ms, FOV=25.6 mm for 46 slices. Writing block: 20 s, rest block: 20 s, total: 5 blocks×6 runs=30 blocks per fMRI. fMRI images were preprocessed using fMRIPrep as detailed herein. FMRIs with excessive head movements (defined as mean frame-wise displacement >0.5 mm) were excluded from the study.

MRI signal analysis: Pre-processed pre-TMS fMRIs from were input into FEAT analysis in FSL software version 6.0 (FMRIB, Oxford, UK) to generate a subject level statistical map. A general linear model in which writing task timing (block design) was convolved with a double-gamma hemodynamic response function to generate the statistical brain map. To account for head motions, fMRIprep reported regressors (CSF, white matter, framewise displacement, and motion outliers) were regressed out from each statistical map. Spatial smoothing with a Gaussian kernel of full-width half-maximum of 5 mm and temporal high pass filter cutoff of 100 seconds was applied during the FEAT analysis. A participant's statistical brain map representing the brain (BOLD) activity during the writing task relative to rest on fMRI was then generated and used for TMS targeting. A WC group-level statistical map for the writing-based task-fMRI was also computed by importing all WC participants' statistical brain maps from their baseline task-fMRI into a mixed-effects FLAME1 model.

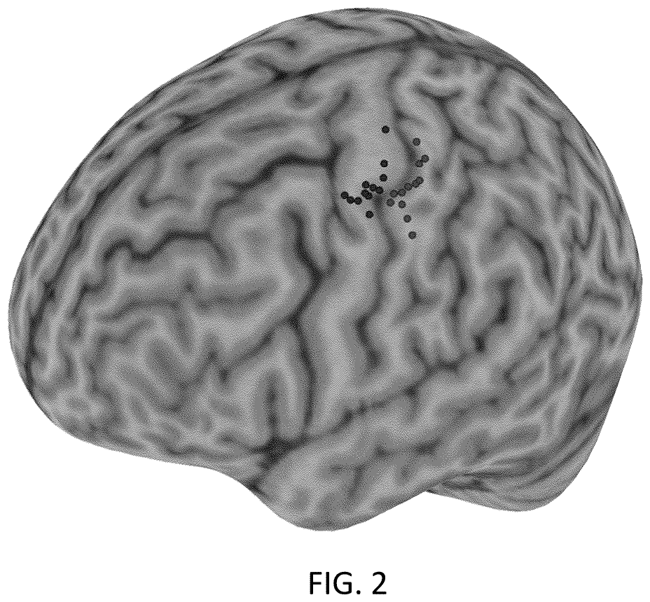

Personalized TMS target selection: For each TMS study participant, a two-voxel cortical brain mask was generated for TMS targeting to premotor and primary motor cortices (PMC), and for TMS targeting to primary somatosensory cortex (PSC). To constrain the stimulation target to the PMC and PSC regions, each participant's statistical brain map was overlayed on the WC group statistical brain map, and anatomical masks for precentral (for PMC) and postcentral gyrus (for PSC) from the Harvard-Oxford MNI atlas and the participant's anatomical scan. Two consecutive voxels in the anatomic region of left PMC and PSC, with peak activation in the WC participant, and the WC group statistical brain maps and within 1 cm from the scalp surface were selected as the fMRI-guided PMC and PSC target for TMS delivery (FIG. 1, target selection panel, red sphere represents PSC target). The fMRI guided cortical brain masks were then used to perform prospective electric field (E-field) modeling on each participant's scalp as detailed herein. The purpose of the modeling was to identify the TMS coil position and orientation on the participant's scalp that would maximize the directional E-field in the cortical target of interest perpendicular to the closest gyral wall. This optimal coil setup was then used online with the patient's T1 to localize and visualize the TMS target in the neuronavigation system (Brainsight, Rogue Research, Canada, version 2.4.9). The final personalized TMS targets at left PMC and left PSC for the 12 WC participants are shown overlayed on the MNI brain (FIG. 2).

TMS stimulation: Eligible participants received three TMS visits. The three TMS visits consisted of 10 Hz rTMS to PSC, 10 Hz rTMS to PMC, and Sham rTMS to PMC (FIG. 1, rTMS panel). Each rTMS visit was separated by a minimum of one week to allow signal washout. To negate any order effect, each participant was randomized to one of six possible orders for the three TMS conditions. All TMS was performed using an A/P Cool B65 coil attached to a MagVenture R30 device (MagVenture, Farum, Denmark). During each TMS visit, participants first completed an optimal motor cortex localization and motor threshold calculation as detailed herein. The active TMS paradigm delivered to each cortical target was 25 trains applied at 10 Hz rTMS with biphasic pulses and an inter-train interval of 10 seconds at 90% resting motor threshold (RMT) for a total of 1000 pulses delivered in a single block (˜five minutes), while participants were in sitting in a recliner. To prime the motor network implicated in focal hand dystonia and circumvent concerns that delivering TMS concurrently during a writing task would compromise stimulation accuracy, an interleaved approach of writing task and brain stimulation was designed. Specifically, each stimulation block was preceded by a writing block (˜five minutes) in which participants performed a sentence copying task. A total of four blocks of TMS alternated with four writing blocks were performed (total 4000 pulses per TMS visit) (FIG. 1, rTMS panel). To perform sham stimulation, the same AP coil was used in placebo mode, which produced clicking sounds and somatosensory sensation from scalp electrodes similar to the active mode but without a significant electric field induced in the brain. As previously reported, this type of stimulation allows participants to stay blinded during the experiment.

Retrospective TMS coil deviations: During each TMS block, data on the experimental TMS coil location and orientation was recorded every 500 ms in the neuronavigation system and snapped to scalp reconstruction mode prior to exporting it. Data was then imported into SimNIBS software (version 2.0.1/3.2.6) to calculate the deviations from the intended TMS coil position and orientation using the retrospective Targeting and Analysis Pipeline (TAP). The TMS coil placement (position and orientation) data were first extruded outwards along the scalp normal by adjusting it for the participant's recorded hair thickness for each TMS visit. These TMS coil placement data were then used to compute the coil placement deviation from the optimized coil setup constructed during the prospective E-field modeling reported herein. The deviations in the TMS coil placement were calculated in the normal and tangential planes and reported as changes in distance (mm) and angle (degrees). The direct distance between the actual and optimized target was also calculated, as previously reported. Due to technical issues with the neuronavigation software, the experimental TMS coil location and orientation during one TMS block was excluded from one participant's sham-TMS visit.

TMS induced behavior changes: During each TMS visit, participants performed a behavioral writing assay before and after each four-block TMS session (FIG. 1, behavior assessment panels). The behavioral assay consisted of participants using a sensor-based pen on a digital tablet (MobileStudio Pro 13; Wacom Co, Ltd, Kazo, Japan). Participants copied a holo-alphabetic sentence ten times in a writing software (MovAlyzer, Tempe, AZ, USA). The sensor-based pen recorded the x, y, and z positions and the time function of the participants' writings. The writing software then transformed the writing samples' position parameters and time functions using a Fast Fourier transform algorithm to calculate the kinematic features automatically. A previously detailed analysis of these kinematic writing measures showed that the sum of acceleration peaks in a single sentence (henceforth peak accelerations) (FIG. 1, behavior assessment panel, black circles) demonstrated high diagnostic potential (sensitivity, specificity, and intra-participant reliability), and associated with patient reported dystonia and disability scales. In this study, the peak accelerations measure was used as the primary behavioral outcome measure. Participants performed the behavioral assessment before and after each TMS session. To minimize learning across the three TMS visits, a different holo-alphabetic sentence was used for each sequential visit. The three sentences were: Pack my box with five dozen liquor jugs; The quick brown fox jumps over the lazy dog; Jinxed wizards pluck blue ivy from the big quilt. To measure the change in peak acceleration, each participant's post-TMS measure was normalized by the mean of their pre-TMS measure using the following equation: [(Post-TMS peak accelerations per sentence)/(mean peak accelerations for all ten sentences pre-TMS)]*100. Higher measures of peak accelerations represent greater writing dysfluency and worsening dystonia. The standard TMS adverse events survey and secondary outcome measures of clinician-rated and participant-reported dystonia scales were also collected before and after each TMS session as detailed herein.

TMS induced fMRI changes: After each TMS visit, participants completed a task-based fMRI. The fMRI task design, acquisition settings, preprocessing and run level analyses are detailed in method above. To compare changes in BOLD activity across the three TMS conditions, 4D timeseries data were extracted from each fMRI run level FEAT directory using brain masks for regions of interests. The timeseries data for each region of interest was z-scored across runs and analyzed as on-block and off-block writing epochs. To perform functional connectivity analysis, the extracted 4D time series data for each region of interest was correlated pairwise using Pearson's correlation (R) and Fisher z-transformed.

Brain parcellation and ROI extraction: To compare BOLD activity and functional connectivity analysis across TMS conditions, brain masks corresponding to regions of the motor network previously identified as abnormal in the writing motor circuit of WC dystonia were used in this study. Specifically, an ROI was included if it was reported in at least two of these five isolated sporadic dystonia studies. Anatomical brain masks were prepared using the Harvard-Oxford MNI brain atlas for the following subcortical regions: left caudate (CAU), left putamen (PUT), left globus pallidus (PAL), left thalamus (THL), left subthalamic nucleus (STN) and left substantia nigra (SN). Since cortex and cerebellum are large brain regions, brain masks for these regions were made using the publicly available Dictionaries of Functional Modes (DiFuMo) brain atlas. DiFuMo is a fine grain atlas that parcellated the brain into functional regions of 1024 components, based on data from 15,000 statistical brain maps spanning 27 studies. To select DiFuMo brain masks for left superior parietal cortex (SPC), left inferior parietal cortex (IPC), left supplementary motor area (SMA) and right cerebellar lobules VI and VIII (CBL), MNI coordinates from prior neuroimaging studies in dystonia were used. For PSC and PMC, each participant's personalized TMS target was used as the center to prepare a custom 5 mm spherical mask.

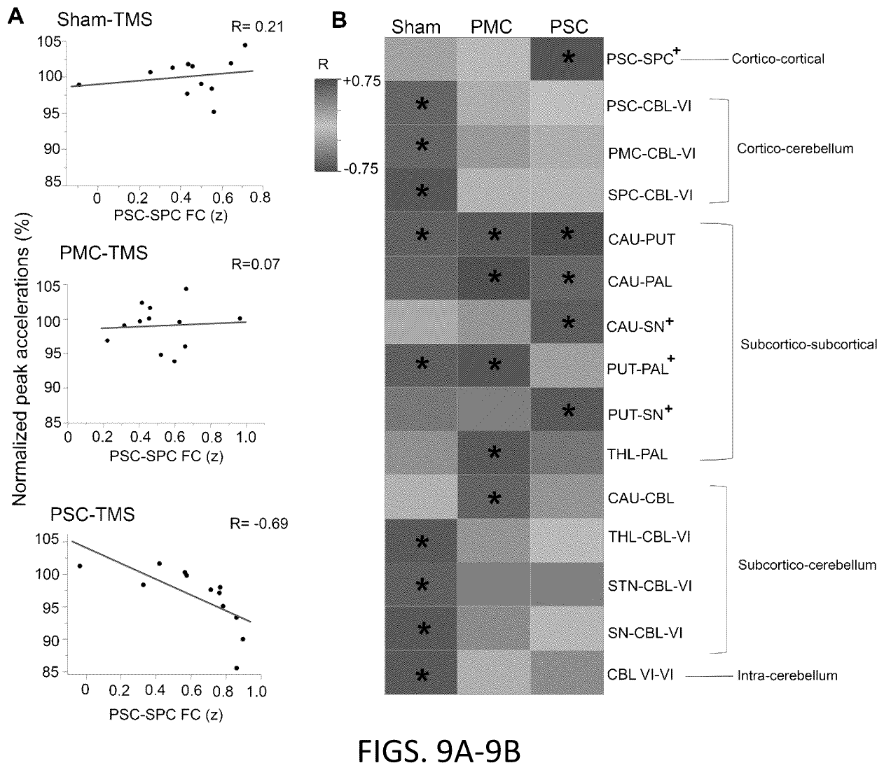

Statistical Analyses: Because the study was a cross-over design with multiple visits and measures, a Mixed-effects Model for Repeated Measures (MEMRM) statistical analysis was used to compare differences in data within participants across the three TMS conditions. For the measure of TMS coil deviations, the MEMRM covariate was TMS condition and since all p-values >0.05, no multiple comparison correction was performed. For the clinician rating scales, the covariates were TMS condition, TMS visit, and clinician rater. For patient rating scales, the covariates were TMS condition, and TMS visit. Since all p-values >0.05, no multiple comparison correction was performed. For the measure of peak accelerations behavior, the covariates for MEMRM were TMS condition, visit, and interaction of TMS condition*visit. Differences across participants for each data set were accounted for by including participant as a random effect variable. Behavior data was adjusted for multiple comparison using Tukey-Holm-Sidak correction with p<0.05 considered significant. For BOLD activity analysis, BOLD activity from 13 brain regions (PMC, PSC, SPC, IPC, SMA, CAU, PUT, PAL, THL, STN, SN, CBL VI, CBL VIII) labeled as motor network were extracted and statistically analyzed using MEMRM. Changes in BOLD activity during the on-block and off-block of writing were analyzed separately. The dependent variable was BOLD activity for each region. The covariates were TMS condition, visit and interaction of TMS condition*visit. BOLD activity analysis was corrected for 60 MEMRM tests using the FDR correction method of Benjamini-Hochberg and p<0.05 considered significant. For functional connectivity, the 13 brain regions were correlated pairwise across the motor network. To focus on clinically meaningful differences induced by TMS, functional connectivities (FC) that showed at least more than minimal effect sizes (defined as Cohen's D>10.21) between active and sham conditions were analyzed using MEMRM test with a setup similar to BOLD activity analysis. FC data were FDR corrected for 126 FC tests for PSC vs. Sham and 166 FC tests for PMC vs. Sham with p<0.05 considered significant. To perform BOLD activity-behavior correlations within participant, BOLD activity from brain regions reported in Table 3 were correlated within participant with their measure of normalized peak accelerations behavior using Pearson's (R). BOLD activity-behavior correlations (R) greater than or equal to 0.6 for at least one TMS condition were presented. An exploratory posthoc analysis was also performed to compare differences in FC-behavior correlation across the three TMS conditions. FC-behavior relationships in the motor network with R-value greater than or equal to 10.61 for at least one TMS condition were identified and presented in a heatmap (FIG. 9B). Using the correlations in FIG. 9B, a subset analysis was performed to statistically compare the FC-peak accelerations correlations that differentiated the effective PSC stimulation site from the non-effective PMC and sham conditions. To evaluate for statistical differences, a generalized linear regression analysis was performed for each FC-behavior relationship that differentiated PSC-TMS from the other two conditions. The dependent variable was peak accelerations behavior, the covariates were FC, TMS condition, and interaction term (FC*TMS condition). P-values for the interaction term for PSC-TMS (PSC-TMS condition*FC) across the selected FCs were corrected for multiple comparisons using the Benjamini-Hochberg method with p<0.1 considered significant.

Double blinding: To blind study participants, participants were told that they will receive TMS at three visits, each at a different brain region and/or TMS intensity. To blind the research staff delivering TMS, the TMS scalp targeting file was void of any identifiers about the cortical location. Prior to each TMS session, the research staff stepped outside the room (and the participant was asked to close their eyes) while the study PI reoriented the AP TMS coil to placebo or active mode. The study PI then covered the TMS machine with a white sheet for the whole research visit to maintain the blinding for the research staff and study participant.

fMRI data preprocessing: fMRI images were preprocessed using fMRIPrep. fMRIPrep is an automated pipeline that performs brain extraction, head motion estimation, distortion correction, slice timing correction, intra-participant registration, and spatial normalization to the Montreal Neurological Institute (MNI) stereotaxic space. fMRIPrep also estimates scan quality for each fMRI run by reporting multiple regressors, including white matter, CSF, framewise displacement, and motion outliers.

Prospective electric field modeling: To perform E-field modeling, each participant's structural T1-weighted scan was utilized to construct a computational head model using the mri2mesh pipeline of SimNIBS (version 2.0.1/3.2.6). The fMRI-based TMS target was then processed in the targeting and analysis pipeline (TAP) to be registered to the space of the head model. In a circular scalp area (2.5 cm radius) above the TMS target, a search over candidate coil setups was conducted in TAP to find the optimal coil setup that maximizes the directional E-field in the cortical target of interest perpendicular to the closest gyral wall. This search iterated over many candidate coil setups in steps of 1 mm and 1° increments for coil center and orientation, respectively. TAP computed an optimal coil placement for a total of 16 different hair thicknesses, prospectively ranging from 0 to 7.5 mm in intervals of 0.5 mm. The hair thickness of the participant at the TMS scalp target was measured on the day of the TMS visit. Out of the 16 hair thicknesses, the numerically closest to the measured value was chosen, and its associated optimal coil setup was utilized in the session with Brainsight. The coil holder was instructed to precisely maintain the optimal scalp placement of the coil relative to the TMS target by adjusting for (head) movements during the entire duration of the TMS intervention.

Motor cortex localization and motor threshold calculation: To identify the optimal motor cortex position of the TMS coil to activate the right first dorsal interosseous muscle, single TMS pulses were delivered while the participant was at rest. The motor cortex position was identified as the position that elicited the largest motor evoked potentials with the least intensity of the maximum stimulator output. The selected motor cortex position was used to calculate the resting motor threshold (RMT). Resting motor threshold (rMT) was defined as the TMS pulse intensity producing, on average an MEP of 50 V peak-to-peak amplitude, using a maximum likelihood estimator (TMS Motor Threshold Assessment Tool, MTAT 2.0 (clinicalresearcher.org/software).

TMS adverse events survey: Participants were asked to rate any adverse effects of TMS before and immediately after each TMS session. Specifically, they were asked if they experienced headache, neck pain, scalp pain, seizure, hearing impairment, cognitive impairment, trouble concentrating, mood change, or any other symptoms. Participants rated the adverse effect on an ordinal scale of absent, mild, moderate, or severe.

TMS-induced changes in clinician-rated and participant-reported scales: Participants were video recorded from the neck down (focused on their right arm) during the pre-TMS and post-TMS writing assessments. Movement Disorder clinicians rated the videos using the Burke-Fahn-Marsden (BFM) dystonia rating scale and Writer's Cramp Rating Scale (WCRS). Clinicians were blinded to the TMS conditions of the WC subjects and provided literature on the rating scales to establish concordant ratings. The two clinicians' inter-rater reliability score was measured using the intraclass correlation coefficient and observed to be 0.45 for the BFM scale and 0.76 for WCRS. WC participants also self-reported their disabilities using two rating scales: the BFM disability scale and the Arms Dystonia and Disability Scale (ADDS). ADDS was scored on a 0-100% scale, with 100% denoted as normal and 0% for severe disability. Since the present study is on focal hand dystonia, for the BFM dystonia scale, clinicians were asked to rate the dystonia severity only in the right arm, and participants rated BFM disability only for handwriting. BFM handwriting disability was scored between 0-4, with 4 being severe. To measure the effect of TMS condition, the absolute change (Post-TMS minus Pre-TMS) in rating scale within participant for each TMS condition was calculated and used for statistical analyses.

### 5. EXAMPLES

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

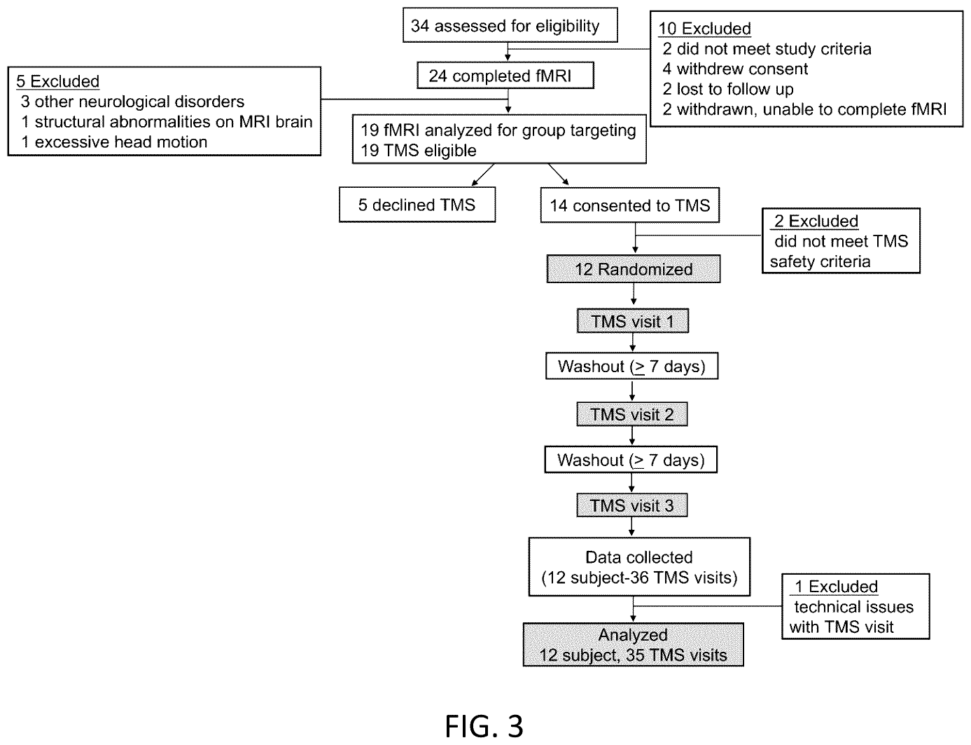

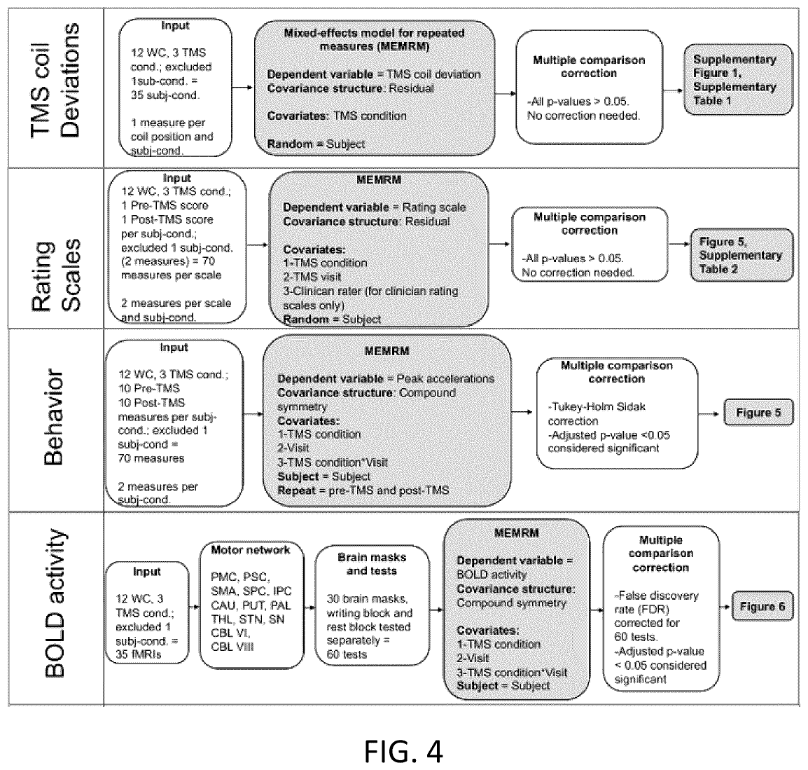

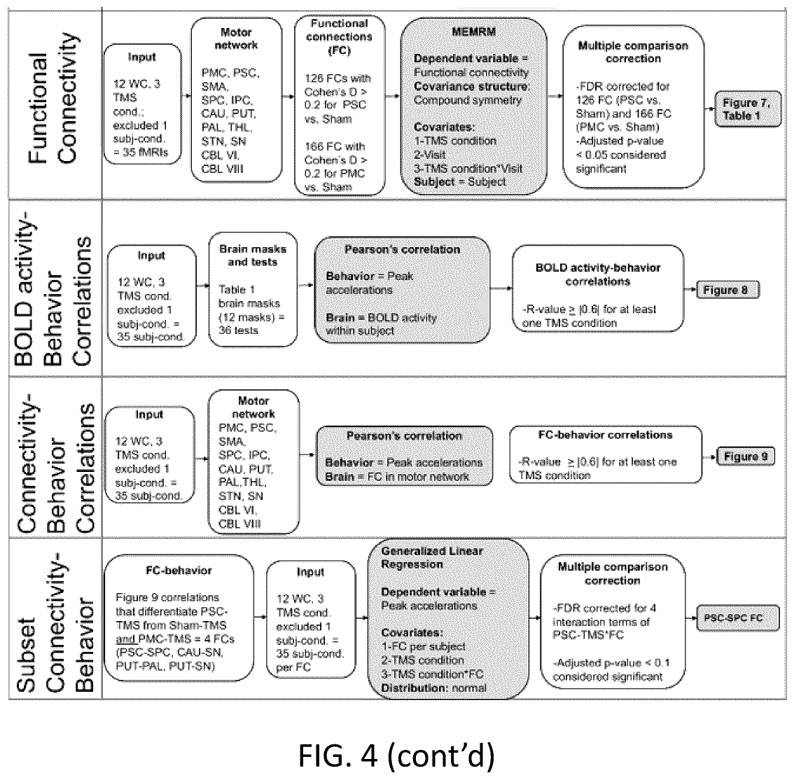

Clinical characteristics: Thirty-four participants were assessed for study eligibility (FIG. 3). Of those assessed for eligibility, 24 WC participants met the inclusion/exclusion criteria to participate in the TMS study and completed a baseline fMRI before the first stimulation visit. The baseline fMRI from five participants were excluded due to other neurological disorder, structural abnormalities on MRI brain or excess head motion. FMRIs from 19 WC participants were then used to identify a group targeting approach for TMS. Of these 19 participants, 14 consented to participate in the TMS research visits. Two participants who consented to TMS visits were taking a medication that increased the risk of seizures and, therefore, were excluded from undergoing TMS. Twelve WC participants (11/1 males/female; mean age 55 [SD 12.91] years) completed all three TMS visits. Due to technical issues during data collection, one participant's TMS visit (Sham) was removed from data analysis. Thus, data from 12 WC participants and 35 TMS visits (12 participants×3 conditions −1 visit=35) were used for all analyses in this study. None of the 12 WC participants reported any TMS adverse side effects. The mean symptom duration for the 12 WC participants was 16.4 years [SD 15.54].

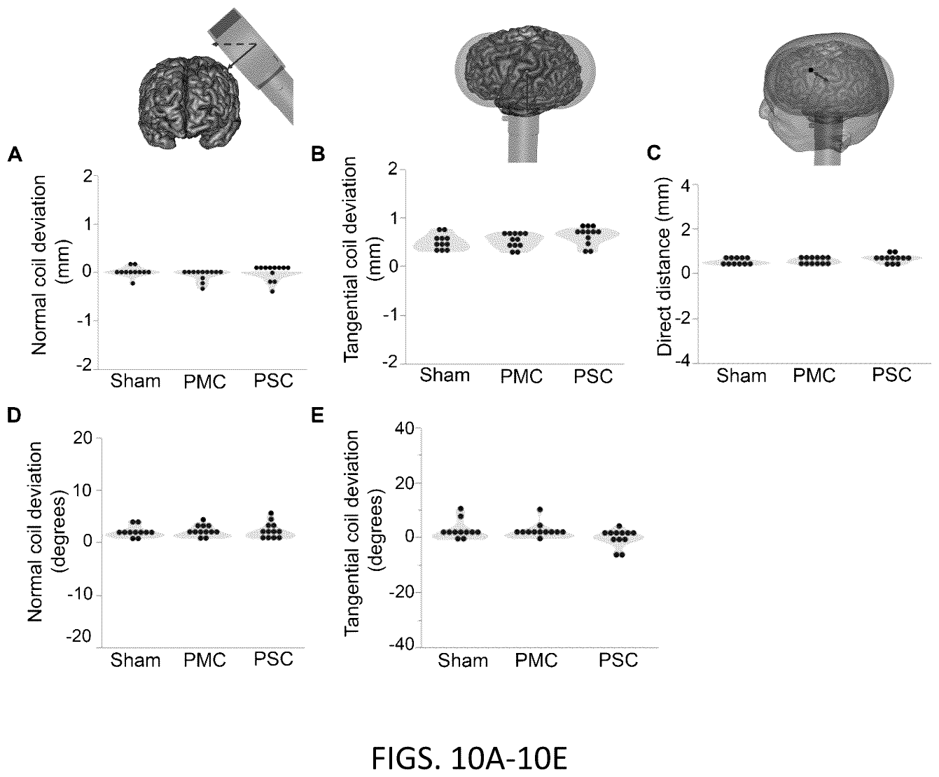

No differences in TMS technical delivery: To evaluate the technical delivery of TMS, the position and orientation of the TMS coil during the three conditions were analyzed retrospectively. There were no significant differences in the position or orientation of the TMS coil across the three conditions (FIG. 10; Table 1). Therefore, TMS delivery across the three conditions was technically comparable.

TABLE 1
A comparison of the deviations in coil position
and orientation for the three TMS conditions.

| Mean Coil | | Difference | | SE | | | |
|---|---|---|---|---|---|---|---|
| Deviations | TMS condition | (mm or deg) | | (mm or deg) | | t-ratio | p-value |
| Normal | PSC vs. Sham | −0.07 | mm | 0.04 | mm | −1.79 | 0.19 |
| Deviation (mm) | PMC vs. Sham | −0.07 | mm | 0.04 | mm | −1.63 | 0.25 |
| | PSC vs. PMC | 0.006 | mm | 0.04 | mm | 0.15 | 0.98 |
| Tangential | PSC vs. Sham | 0.09 | mm | 0.04 | mm | 2.02 | 0.13 |
| Deviation (mm) | PMC vs. Sham | 0.01 | mm | 0.04 | mm | 0.32 | 0.94 |
| | PSC vs. PMC | −0.08 | mm | 0.04 | mm | −1.74 | 0.21 |
| Direct | PSC vs. Sham | 0.10 | mm | 0.05 | mm | 1.96 | 0.14 |
| Distance (mm) | PMC vs. Sham | 0.02 | mm | 0.05 | mm | 0.4 | 0.91 |
| | PSC vs. PMC | −0.08 | mm | 0.05 | mm | −1.60 | 0.26 |
| Normal | PSC vs. Sham | 0.12 | deg | 0.24 | deg | 0.53 | 0.85 |
| Deviation (deg) | PMC vs. Sham | 0.02 | deg | 0.24 | deg | 0.09 | 0.99 |
| | PSC vs. PMC | −0.1 | deg | 0.23 | deg | −0.44 | 0.89 |
| Tangential | PSC vs. Sham | −2.73 | deg | 1.42 | deg | −1.91 | 0.15 |
| Deviation (deg) | PMC vs. Sham | −0.38 | deg | 1.42 | deg | −0.27 | 0.95 |
| | PSC vs. PMC | 2.34 | deg | 1.39 | deg | 1.67 | 0.23 |

SE: Standard Error

### Example 2

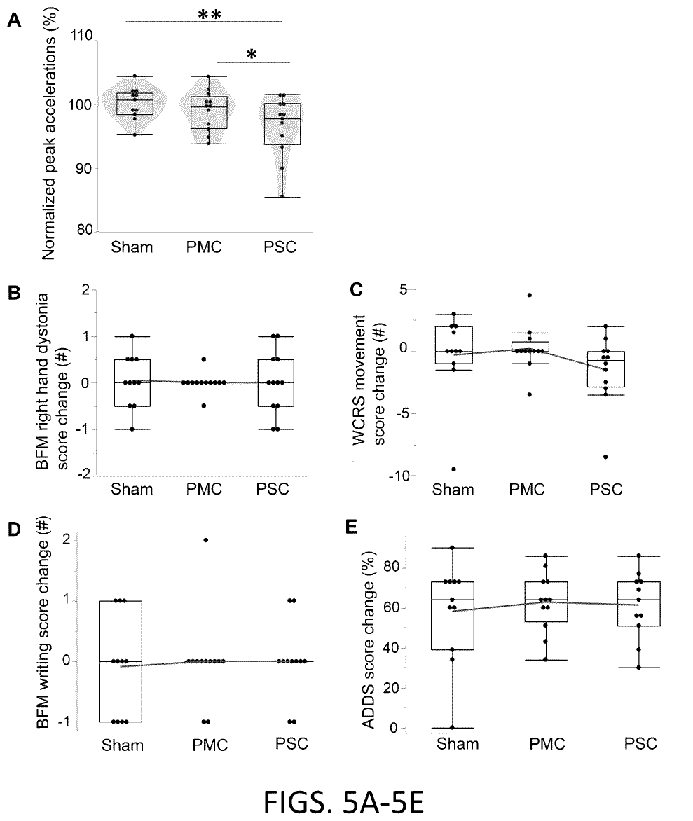

10 Hz rTMS to PSC, but not PMC, improved writing dysfluency: In a within-participant comparative design, in which all participants received stimulation to both sites and at the same frequency, it was found that 10 Hz rTMS to PSC significantly decreased writing dysfluency compared to Sham-TMS (FIG. 5A) [PSC: mean 96.43, SE 1.39; Sham: mean 100.06, SE 0.76; PSC vs. Sham: −1.73, SE: 0.41, t(21): −4.22, p=0.001] and PMC−TMS [PMC: mean 99.00, SE 0.90; PSC vs. PMC: −1.28, SE 0.40, t(21): 3.23, p=0.012]. TMS to PMC did not show significant differences in writing dysfluency compared to Sham [IPMC vs. Sham: −0.45, SE 0.41, t(21): −1.09, p=0.639]. These results confirm prior studies that TMS can modify behavior in WC dystonia and show the first results using 10 Hz frequency, within-participant site comparisons, and delivery under a task-primed brain state. Across the clinician rating (BFM right arm dystonia, and WCRS movement score) and participant reported scales (BFM writing score, and ADDS), there were small but consistent improvements in dystonia symptoms after PSC-TMS compared to Sham (FIGS. 5B-5E; Table 2), “Difference” column, positive values represent improvement, and negative values represent worsening dystonia). However, the effect sizes of these categorical rating scales were small with large variability resulting in no statistical differences across the three TMS conditions.

TABLE 2
Comparison of TMS-induced changes in clinician and subject rating scales.

| | | TMS | Difference | SE | | |
|---|---|---|---|---|---|---|
| Rater | Rating Scale | condition | (#) | (#) | t-ratio | p-value |
| Clinicians | BFM dystonia | PSC vs. Sham | 0.07 | 0.13 | 0.54 | 0.59 |
| | right hand | PMC vs. Sham | 0.05 | 0.13 | 0.42 | 0.68 |
| | | PSC vs. PMC | −0.02 | 0.13 | −0.13 | 0.90 |
| | WCRS | PSC vs. Sham | 1.24 | 0.67 | 1.85 | 0.08 |
| | movement score | PMC vs. Sham | 0.03 | 0.67 | 0.05 | 0.96 |
| | | PSC vs. PMC | −1.2 | 0.66 | −1.83 | 0.08 |
| WC | BFM writing | PSC vs. Sham | 0.03 | 0.19 | 0.18 | 0.86 |
| subjects | score | PMC vs. Sham | −0.05 | 0.19 | −0.27 | 0.79 |
| | | PSC vs. PMC | −0.08 | 0.18 | −0.46 | 0.65 |
| | ADDS total | PSC vs. Sham | 2.28 | 2.22 | 1.03 | 0.31 |
| | score | PMC vs. Sham | 2.74 | 2.22 | 1.24 | 0.23 |
| | | PSC vs. PMC | 0.46 | 2.14 | 0.21 | 0.83 |

SE: Standard Error

### Example 3

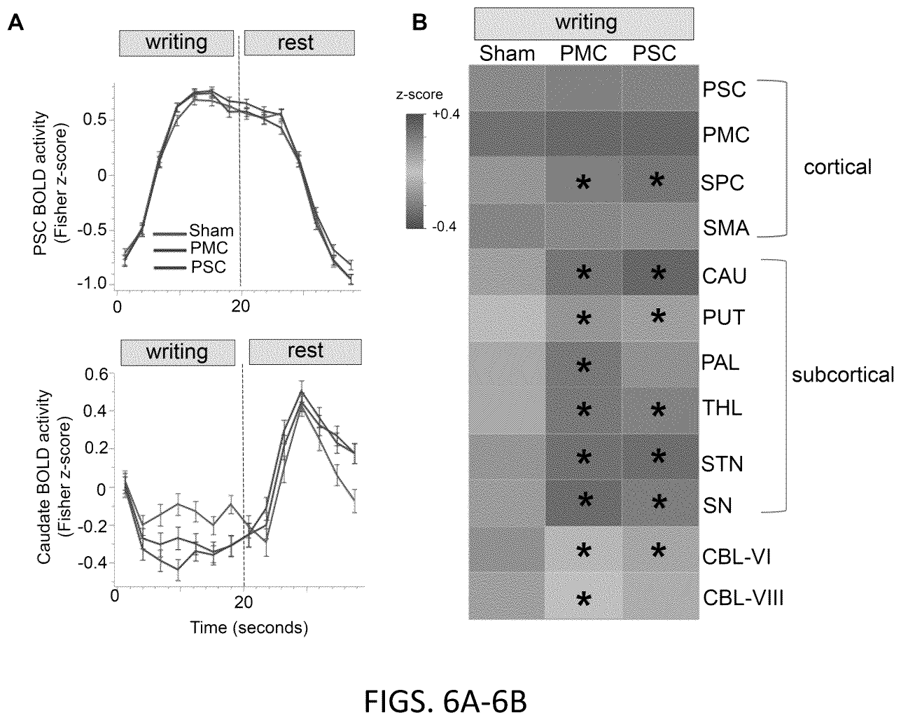

10 Hz rTMS to either PMC or PSC decreased subcortical activity in the motor network compared to sham-TMS. Considering the differential effect of the stimulation site on behavioral outcomes, experiments were conducted to examine how active TMS at these two target sites affected brain BOLD activity during a writing task relative to both rest and sham-TMS condition (FIG. 6A). Across the motor network, active stimulation at either of the two TMS target sites showed similar patterns of brain activation during the writing task compared to sham-TMS (FIG. 6B). Specifically, 10 Hz rTMS decreased subcortical brain activity and increased BOLD activity at the superior parietal cortex during writing compared to sham-TMS. In the cerebellum, the brain activation pattern during writing varied by the active TMS target site. Active PMC-TMS decreased BOLD activity in lobules VI and VIII while active PSC-TMS decreased BOLD activity in lobule VI only during writing compared to sham-TMS. Overall, these results suggest that cortically delivered 10 Hz rTMS decreased deep brain activity in the motor network compared to sham-TMS with an overall activation pattern during writing that was similar across the two stimulation sites. Changes in BOLD activity after 10 Hz rTMS, therefore, do not fully explain the differential effect of stimulation site on behavioral outcomes.

### Example 4

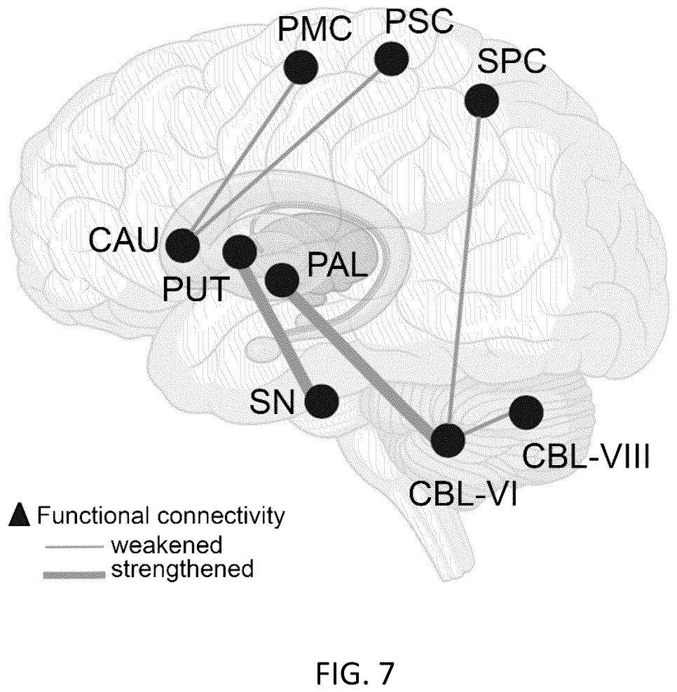

PSC-TMS modified subcortical connectivity, distinct from PMC-TMS and sham-TMS. Experiments were conducted to investigate whether the behavioral outcome differences between the stimulation sites also corresponded to changes in functional connectivity (FC) after TMS. FIG. 7 illustrates the FC changes induced by PSC-TMS compared to sham-TMS. In general, PSC-TMS weakened cortico-striatal FC compared to sham-TMS (thin lines: PMC-CAU, PSC-CAU), cortico-cerebellar FC (SPC-CBLVI), and intra-cerebellar FC (CBL VI-VIII). PSC-TMS also strengthened striato-cerebellar FC (thick lines: PAL-CBL-VI) and striato-nigral FC (PUT-SN) compared to sham-TMS (Table 3). There were no FCs that showed significant differences between PMC-TMS compared to sham-TMS. Overall, these findings demonstrate two important points. 10 Hz rTMS to PSC interleaved with writing task predominantly changed subcortical FC compared to sham-TMS. Second, changes in FC induced by TMS may explain the differential effect of stimulation site on behavioral outcomes.

TABLE 3
Changes in functional connectivity induced by PSC-
TMS compared to sham-TMS in the motor network.

| | | | | Mixed-effects Model for | | | |
|---|---|---|---|---|---|---|---|
| | | | | Repeated Measures | | | |
| | | FC mean z-score | | PSC vs. Sham | | | |
| Brain | Functional | (SE) | | Mean FC | | | |
| Regions | connections | Sham | PSC | difference | SE | t-ratio | p-value* |
| Cortical- | PMC-CAU | 0.49 (0.03) | 0.33 | −0.17 | 0.04 | −4.49 | 0.015 |
| striatal | | | (0.03) | | | | |
| | PSC-CAU | 0.23 (0.03) | 0.10 | −0.13 | 0.03 | −4.17 | 0.015 |
| | | | (0.03) | | | | |
| Cortico- | SPC-RCBL-VIII | 0.44 (0.03) | 0.27 | −0.15 | 0.05 | −3.38 | 0.048 |
| cerebellar | | | (0.04) | | | | |
| Subcortico- | PAL-CBL-VI | 0.13 (0.04) | 0.22 | +0.17 | 0.05 | 3.61 | 0.041 |
| subcortical | | | (0.04) | | | | |
| | PUT-SN | 0.48 (0.04) | 0.59 | +0.16 | 0.04 | 3.53 | 0.042 |
| | | | (0.03) | | | | |
| Intra- | R-CBL VI-VIII | 0.51 (0.04) | 0.40 | −0.18 | 0.04 | −4.28 | 0.015 |
| cerebellar | | | (0.05) | | | | |
| | R-CBL VI-VIII | 0.45 (0.03) | 0.30 | −0.20 | 0.05 | −4.12 | 0.015 |
| | | | (0.05) | | | | |

PMC: premotor cortex; CAU: caudate, PSC: primary somatosensory cortex; SPC: superior parietal cortex; R-CBL-VIII: right cerebellum lobule VIII; PAL: pallidum; PUT: putamen; SN: substantia nigra; R-CBL VI: right cerebellum lobule VI; SE: Standard Error, *p-values after MEMRM and FDR correction.

### Example 5

TMS-induced changes in PSC and SPC BOLD activity were associated with reduction in writing dysflZuency. Experiments were conducted to investigate whether there was a relationship between TMS-induced brain activation and behavior changes and if this relationship was dependent on the stimulation site. A BOLD activity-behavior correlational analysis was performed for all brain regions in Table 3 that showed significant differences in functional connectivity between PSC-TMS and sham-TMS. Among these brain regions, PSC and SPC BOLD activity correlated with behavior of peak accelerations. Specifically, increase in PSC BOLD activity after PSC-TMS was associated with reduction of peak accelerations behavior in WC participants (R=−0.84, p=0.02) (FIG. 8). In contrast, there were no correlations observed between PSC BOLD activity and behavior after PMC-TMS (R=−0.39, p=0.75) or sham-TMS (R=0.15, p=0.76). In contrast, increase in SPC BOLD activity correlated with increase in peak accelerations behavior after Sham-TMS in WC participants (R=0.74, p=0.01). This SPC BOLD-behavior correlation was not observed after PMC-TMS (R=−0.19, p=0.55) or PSC-TMS (R=−0.15, p=0.64). Collectively, these results suggest that TMS induced changes in the association between brain activity and behavioral measures were dependent on the stimulation site and TMS induced activation of PSC and SPC after PSC-TMS were associated with reduction in writing dysfluency.

### Example 6

PSC-TMS induced reorganization of motor network connectivity was associated with reduced writing dysfluency. Additionally, post-hoc analysis was conducted to determine whether there was an association between TMS-induced changes in functional connectivity in the motor network and TMS-induced changes in behavior and if this association was dependent on the stimulation site. From this analysis, three observations were made (FIG. 9). First, under Sham-TMS condition (control condition), nine significant FC relationships with writing dysfluency were identified with majority of them in connection to the cerebellum (cortico-cerebellum, subcortical-cerebellum, intra-cerebellum). Second, compared to sham, correlations between writing dysfluency and FC involving the cerebellum were no longer present following 10 Hz rTMS to either PMC or PSC. The loss of FC-behavior correlations in these regions was observed to a greater extent after PSC-TMS than PMC-TMS. Third, using the FC-peak accelerations correlations in FIG. 9B, a subset analysis was performed to statistically compare the FC-peak accelerations relationships that differentiated the effective PSC stimulation site from the non-effective PMC and sham conditions. PSC-TMS differed from the other two stimulation sites in four FC-behavior correlations (PSC-SPC, CAU-SN, PUT-PAL, PUT-SN) that spanned cortical and subcortical brain regions (FIG. 9B, indicated by “+” sign). Of these four correlations, PSC-TMS significantly differed from sham-TMS in the PSC-SPC FC-behavior association (PSC vs. Sham: −14.6, p=0.075, generalized linear regression, Benjamini-Hochberg adjustment for multiplicity) (FIG. 9A). Collectively, these results demonstrated that reduced writing dysfluency after PSC-TMS may be mediated by loss of functional connectivity-behavior associations to the cerebellum, and/or gain of functional connectivity-behavior associations to cortical and subcortical brain regions.

Overall, TMS target comparison demonstrated that 10 Hz rTMS to primary somatosensory cortex but not premotor cortex significantly changed functional connectivity and markedly redistributed functional connectivity-behavior associations that spanned cortical and subcortical regions of the motor network.

What is claimed is:

1. A method of treating dystonia, the method comprising administering repetitive transcranial magnetic stimulation (rTMS) to a subject, wherein the rTMS targets a cortical region of the subject's brain, and wherein administering the rTMS treats at least one symptom of dystonia in the subject.

2. The method of claim 1, wherein the rTMS is administered to the premotor cortex (PMC).

3. The method of claim 1, wherein the rTMS is administered to the primary somatosensory cortex (PSC).

4. The method of claim 1, wherein the rTMS is administered to the parietal cortex (PC).

5. The method of claim 1, wherein the rTMS is administered at a frequency ranging from about 5 Hz to about 20 Hz.

6. The method of claim 1, wherein the rTMS comprises administering about 10 stimulation trains to about 50 stimulation trains.

7. The method of claim 6, wherein the trains are administered at an inter-train interval of about 5 seconds to about 30 seconds.

8. The method of claim 1, wherein the rTMS is administered using a plurality of stimulation blocks, wherein each stimulation block is from about 1 minute to about 10 minutes.

9. The method of claim 8, wherein about 500 pulses to about 2000 pulses are delivered in each stimulation block.

10. The method of claim 9, wherein each pulse is the same phase.

11. The method of claim 8, wherein each stimulation block is interleaved with a motor activity block during which the subject performs or imagines performing a motor task.

12. The method of claim 8, wherein the plurality of stimulation blocks range from about 2 stimulation blocks to about 10 stimulation blocks.

13. The method of claim 8, wherein a total of about 1000 pulses to about 50,000 pulses are delivered to the subject during a single treatment.

14. The method of claim 1, wherein the at least one symptom of dystonia that is treated in the subject comprises at least one of muscle tightness, muscle contraction, muscle pain, decreased coordination, abnormal posture, hand spasms, task-related tremor, and/or motor dysfluency.

15. The method of claim 1, wherein administration of the rTMS to the subject improves at least one functional connection in a motor network subregion, thereby treating the at least one symptom of dystonia in the subject.

16. The method of claim 1, wherein the method further comprises assessing neural activity in the subject before and/or after administering the rTMS.

17. The method of claim 16, wherein the neural activity is assessed using magnetic resonance imaging (MRI) or functional magnetic resonance imaging (fMRI).

18. The method of claim 1, wherein the method further comprises performing rTMS target selection prior to administering rTMS to the subject, wherein the target selection comprises assessing neural activity of the subject during performance of a motor task.

19. The method of claim 18, wherein the neural activity is assessed using magnetic resonance imaging (MRI) or functional magnetic resonance imaging (fMRI).

20. The method of claim 1, wherein the rTMS is administered during more than one treatment session on consecutive days.

21. The method of claim 1, wherein the rTMS is administered during more than one treatment session on non-consecutive days.

22. The method of claim 1, wherein the rTMS is administered during more than one treatment session separated by a period of up to about six months.

23. The method of claim 1, wherein the rTMS is administered during more than one treatment session ranging from about 2 consecutive days to about 10 consecutive days.

24. A method of treating dystonia in a subject, the method comprising:
performing repetitive transcranial magnetic stimulation (rTMS) target selection comprising assessing neural activity of the subject during performance of a motor task using functional magnetic resonance imaging; and
administering rTMS at a frequency ranging from about 5 to about 20 Hz to a target cortical region of the subject's brain identified using the rTMS target selection, wherein administering the rTMS treats at least one symptom of dystonia in the subject.
